SEQUENCE LISTING (3 sequences)

SEQ ID NO: 1 - DNA, 1533 bp, Artificial Sequence
  codon-optimized nucleic acid sequence encoding SIV mac239 gag
atgggggtgaggaactctgtgctgtctggcaagaaggctgatgagctggagaagatcaggctgaggcccaatggcaagaagaagtacatgctgaagcatgtggtgtgggctgccaatgagctggacaggtttggcctggctgagtccctgctggagaacaaggagggctgccagaagatcctgtctgtgctggcccccctggtgcccacaggctctgagaacctgaagtccctgtacaacacagtgtgtgtgatctggtgcatccatgctgaggagaaggtgaagcacacagaggaggccaagcagattgtgcagaggcacctggtggtggagacaggcaccacagagaccatgcccaagacctccaggcccacagccccctcctctggcagggggggcaactaccctgtgcagcagattgggggcaactatgtgcacctgcccctgtcccccaggaccctgaatgcctgggtgaagctgattgaggagaagaagtttggggctgaggtggtgcctggcttccaggccctgtctgagggctgcaccccctatgacatcaaccagatgctgaactgtgtgggggaccaccaggctgctatgcagatcatcagggacatcatcaatgaggaggctgctgactgggacctgcagcacccccagcctgccccccagcagggccagctgagggagccctctggctctgacattgctggcaccacctcctctgtggatgagcagatccagtggatgtacaggcagcagaaccccatccctgtgggcaacatctacaggaggtggatccagctgggcctgcagaagtgtgtgaggatgtacaaccccaccaacatcctggatgtgaagcagggccccaaggagcccttccagtcctacgtggacaggttctacaagtccctgagggctgagcagacagatgctgctgtgaagaactggatgacccagaccctgctgatccagaatgccaaccctgactgcaagctggtgctgaagggcctgggggtgaaccccaccctggaggagatgctgacagcctgccagggggtggggggccctggccagaaggccaggctgatggctgaggccctgaaggaggccctggcccctgtgcccatcccctttgctgctgcccagcagaggggccccaggaagcccatcaagtgctggaactgtggcaaggagggccactctgccaggcagtgcagggcccccaggaggcagggctgctggaagtgtggcaagatggaccatgtgatggccaagtgccctgacaggcaggctggcttcctgggcctgggcccctggggcaagaagcccaggaacttccccatggcccaggtgcaccagggcctgatgcccacagccccccctgaggaccctgctgtggacctgctgaagaactacatgcagctgggcaagcagcagagggagaagcagagggagtccagggagaagccctacaaggaggtgacagaggacctgctgcacctgaactccctgtttgggggggaccagtaa

SEQ ID NO: 2 - DNA, 744 bp, Artificial Sequence
  codon-optimized nucleic acid sequence encoding SIV mac251 nef with a G2A mutation
atggccggagctatttccatgaggcggtccaagccggctggagatctgcgacagaaactcttgcgggcgcgtggagagacttatgggagactcttaggagaggtggaagatggatcctcgcaatccctaggaggattaggcaagggcttgagctcacgctcttgtgagggacagaaatacaatcaggggcagtatatgaatactccatggagaaacccagctgaagaaaaagaaaaattagcatacagaaaacaaaatatggatgatatagatgaggaagatgatgacttggtaggggtatcagtgaggccaaaagttcccctaagagcaatgacttacaaattggcaatagatatgtctcattttataaaagaaaaggggggactggaagggatttattacagtgcaagaagacatagaatcttagacatgtacttagaaaaggaagaaggcatcataccagattggcaggattacacctcaggaccaggaattagatacccaaagacatttggctggctatggaaattagtccctgtaaatgtatcagatgaggcacaggaggatgagaggcattatttaatgcagccagctcaaacttccaagtgggatgacccttggggagaggttctagcgtggaagtttgatccaactctagcctacacttatgaggcatatgctagatacccagaagagttggaagcaagtcaggcctgtcagaggaagaggttagaagaaggctaa

SEQ ID NO: 3 - DNA, 49 bp, Artificial Sequence
  short synthetic poly A signal
aataaaagatctttattttcattagatctgtgtgttggttttttgtgtg

(19) United States
(12) Patent Application Publication
(10) Pub. No.: US 2006/0216272 A1
(43) Pub. Date: Sep. 28, 2006

(54) Therapeutic immunization of hiv-infected individuals

(76) Inventors: Emilio A. Emini, Wayne, PA (US); John W. Shiver, Chalfont, PA (US); Danilo R. Casimiro, Harleyville, PA (US); Daria Hazuda, Doylestown, PA (US); William A. Scheilf, Green Lane, PA (US)

Correspondence Address: MERCK AND CO., INC, P O BOX 2000, RAHWAY, NJ, 07065-0907, US

(21) Appl. No.: 10/571,651

(22) PCT Filed: Sep. 14, 2004
(86) PCT No.: PCT/US04/29844

§ 371 Date: Mar. 13, 2006

Related U.S. Application Data
(60) Provisional application No. 60/504,522, filed on Sep. 18, 2003.

(51) Int. Cl. A61K 48/00

(52) U.S. Cl. 424/93.2; 514/44

(57) ABSTRACT

The present invention provides an improved method for eliciting a therapeutic immune response in an individual infected with human immunodeficiency virus (“HIV”). The method comprises administering an adenoviral vaccine composition expressing an HIV antigen to an individual with controlled viremia. Immunization of infected individuals in this manner elicits a cellular-mediated immune response against the virus that is significant both in the level of the response and the breadth of the response. The therapeutic immune response that ensues is capable of effectively maintaining low titers of virus and, thus, offers the prospect of reducing individual dependency on antiviral therapy.

### CROSS-REFERENCE TO RELATED APPLICATIONS

This application claims the benefits of U.S. provisional application Ser. No. 60/504,522, filed Sep. 18, 2003.

### STATEMENT REGARDING FEDERALLY-SPONSORED R&D

Not Applicable

### REFERENCE TO MICROFICHE APPENDIX

Not Applicable

### FIELD OF THE INVENTION

The present invention discloses an effective means for containing viral replication in HIV-infected individuals with controlled viremia. The method comprises immunization of said individuals with recombinant, replication-defective adenovirus comprising exogenous nucleic acid encoding an HIV antigen.

### BACKGROUND OF THE INVENTION

Human Immunodeficiency Virus (HIV) is the etiological agent of acquired human immune deficiency syndrome (AIDS) and related disorders. HIV is an RNA virus of the Retroviridae family and exhibits the 5′LTR-gag-pol-env-LTR 3′ organization of all retroviruses. The integrated form of HIV, known as the provirus, is approximately 9.8 Kb in length. Each end of the viral genome contains flanking sequences known as long terminal repeats (LTRs).

HIV genes encode at least nine proteins and are divided into three classes; the major structural proteins (Gag, Pol, and Env), the regulatory proteins (Tat and Rev); and the accessory proteins (Vpu, Vpr, Vif and Nef). The gag gene encodes a 55-kilodalton (kDa) precursor protein (p55) which is expressed from the unspliced viral mRNA and is proteolytically processed by the HIV protease, a product of the pol gene. The mature p55 protein products are p17 (matrix), p24 (capsid), p9 (nucleocapsid) and p6. The pol gene encodes proteins necessary for virus replication—reverse transcriptase, protease, integrase and RNAse H. These viral proteins are expressed in a Gag-Pol fusion protein, a 160 kDa precursor protein which is generated via a ribosomal frame shifting. The virally encoded protease proteolytically cleaves the Pol polypeptide away from the Gag-Pol fusion and further cleaves the Pol polypeptide to the mature proteins which provide protease (Pro, P10), reverse transcriptase (RT, P50), integrase (IN, p31) and RNAse H(RNAse, p15) activities. The nef gene encodes an early accessory HIV protein (Nef) which has been shown to possess several activities such as down regulating CD4 expression, disturbing T-cell activation and stimulating HIV infectivity. The env gene encodes the viral envelope glycoprotein that is translated as a 160-kilodalton (kDa) precursor (gp160) and then cleaved by a cellular protease to yield the external 120-kDa envelope glycoprotein (gp120) and the transmembrane 41-kDa envelope glycoprotein (gp41). Gp120 and gp41 remain associated and are displayed on the viral particles and the surface of HIV-infected cells. The tat gene encodes a long form and a short form of the Tat protein, a RNA binding protein which is a transcriptional transactivator essential for HIV replication. The rev gene encodes the 13 kDa Rev protein, a RNA binding protein. The Rev protein binds to a region of the viral RNA termed the Rev response element (RRE). The Rev protein promotes transfer of unspliced viral RNA from the nucleus to the cytoplasm. The Rev protein is required for HIV late gene expression and in turn, HIV replication.

The virally expressed proteins enable the virus to enter the target cell and direct replication of viral RNA for eventual production of additional infectious virus. Gp120 binds to the CD4/chemokine receptor present on the surface of helper T-lymphocytes, macrophages and other target cells in addition to other co-receptor molecules. X4 (macrophage tropic) virus show tropism for CD4/CXCR4 complexes while R5 (T-cell line tropic) virus interact with a CD4/CCR5 receptor complex. After gp120 binds to CD4, gp41 mediates the fusion event responsible for virus entry. The virus then fuses with and enters the target cell, a process followed by reverse transcription of its single stranded RNA genome into double-stranded DNA via a RNA dependent DNA polymerase. The viral DNA, known as provirus, then enters the cell nucleus, where the viral DNA directs the production of new viral RNA within the nucleus, expression of early and late HIV viral proteins, and subsequently the production and cellular release of new virus particles. Recent advances in the ability to detect viral load within the host shows that the primary infection results in an extremely high generation and tissue distribution of the virus, followed by a steady state level of virus (albeit through a continual viral production and turnover during this phase), leading ultimately to another burst of virus load which leads to the onset of clinical AIDS. Productively infected cells have a half life of several days, whereas chronically or latently infected cells have a 3-week half life, followed by non-productively infected cells which have a long half life (over 100 days) but do not significantly contribute to day-to-day viral loads seen throughout the course of disease.

Destruction of CD4 helper T lymphocytes, which are critical to immune defense, is a major cause of the progressive immune dysfunction that is the hallmark of HIV infection. The loss of CD4 T-cells seriously impairs the body's ability to fight most invaders, but it has a particularly severe impact on the defenses against viruses, fungi, parasites and certain bacteria, including mycobacteria.

Effective treatment regimens for HIV infected individuals have become available and are instrumental in the treatment of individuals infected with HIV. Antiviral agents (including but not limited to antiretroviral therapy (“ART”)) which act as inhibitors of HIV replication have proven extremely successful in the treatment of AIDS and similar diseases; effective treatment with antiviral drugs having been reported as decreasing viral load levels by 90% or more within 8 weeks, effecting a continual reduction in viral load to eventual undetectable levels within 6 months. Several classes of antiviral compounds now exist including but not limited to inhibitors of reverse transcriptase (e.g., azidothymidine (AZT) and efavirenz); protease (e.g., indinavir and nelfinavir); and integrase.

Unfortunately, these drugs will not have a significant impact on the disease in many parts of the world. Furthermore, in individuals with these treatment options available, treatment will require long term antiretroviral therapy in order to maintain low levels of virus and, ultimately, prevent viral rebound. For this reason, recent efforts have focused on promoting an immune response in HIV-infected persons whom have received antiretroviral therapy by administering an immunogen(s) to infected individuals. Noted publications employ an HIV antigen as the immunogen and deliver same by DNA administration, administration of a whole killed (gp120-deleted) HIV-1 vaccine, or administration via a pox viral vector (e.g., ALVAC, NYVAC); see, e.g., Hoff and McNamara, 1999 The Lancet 353:1723-1724; and the following patent publications: WO 98/08539; WO 01/08702; WO 01/54701; and WO 02/095005.

To Applicants' knowledge, previously infected HIV persons exhibiting controlled viremia have not been immunized with recombinant, replication-defective adenovirus comprising exogenous nucleic acid encoding an HIV antigen. As disclosed herein, this method can induce very high levels of both virus specific CD8+ and CD4+ T cell responses of a very broad nature. The therapeutic immune response that ensues has the capability of effectively maintaining low titers of virus and, thus, offers the prospect of reducing individual dependency on antiviral therapy. It would be of great import in the battle against AIDS to produce a vaccine regimen of use in HIV-infected individuals which could assist in reviving a strong HIV-specific cellular mediated immune response in infected individuals.

### SUMMARY OF THE INVENTION

The present invention provides an improved method for eliciting a therapeutic immune response in individuals infected with human immunodeficiency virus (“HIV”). The method comprises immunizing infected individuals exhibiting an active control of viremia (whether by means of an active immune response or through treatment with antiviral agents) by administering a recombinant, replication-defective adenovirus comprising exogenous nucleic acid encoding at least one HIV antigen. Immunization in this manner induces a notable increase in virus-specific CD8+ and CD4+ T cell responses of a very broad nature. The therapeutic immune response that ensues has the capability of effectively maintaining low titers of virus and, thus, offers the prospect of reducing individual dependency on antiviral therapy.

Cytotoxic T Lymphocytes (“CTL”) form an essential part of the cellular response of the immune system. In order to elicit CTL immune responses, antigen must be synthesized within or introduced into cells, subsequently processed into small peptides by the proteasome complex, and translocated into the endoplasmic reticulum/Golgi complex secretory pathway for eventual association with major histocompatibility complex (MHC) class I proteins. CD8+ T lymphocytes recognize antigen in association with class I MHC via the T cell receptor (TCR) and the CD8 cell surface protein. Activation of naive CD8+ T cells into activated effector or memory cells generally requires both TCR engagement of antigen as described above as well as engagement of co-stimulatory proteins. Optimal induction of CTL responses usually requires “help” in the form of cytokines from CD4+ T lymphocytes which recognize antigen associated with MHC class II molecules via TCR and CD4 engagement. The instant invention has the capability of inducing both CD8+ and CD4+ responses in individuals infected with HIV in instances where the individuals, prior to or simultaneous with vaccine administration, have effectively contained viral replication, be it through an active immune response on the part of the treated individual or a favorable response to antiviral therapy.

Accordingly, the present invention is drawn to a method for eliciting a cellular-mediated immune response against HIV in an individual infected with HIV, which comprises administering to an individual that has experienced a reduction in HIV viral copy number a recombinant, replication-defective adenovirus comprising exogenous nucleic acid encoding an HIV antigen. This status of having a reduced viral load as compared to some prior time point, whether facilitated or not, is generally referred to herein as “controlled” or “contained”. In preferred embodiments, the viral load has been reduced and is of an order of magnitude of 10,000 viral copies or less; more preferably, of approximately 5,000 copies or less. Preferably, the individual has a CD4+ count of at least 300 cells per ml of plasma; more preferably, above 400 cells per ml of plasma; most preferably, above 500 cells per ml of plasma. It is also preferable that the individual(s) has not as of yet progressed to AIDS. The cause behind a reduction in viral number at the time of immunization is not critical. The reduction can, for instance, be mediated by an innate ability of the immune system to respond to the presence of the virus; a prior immunization which assists the individual in keeping the viral load under control; or treatment with antiviral agents. The antiviral agent(s) can be selected from any compound or therapy capable of effecting a reduction of viral load. The antiviral agent is, preferably, selected from the class of compounds consisting of: a protease inhibitor, an inhibitor of reverse transcriptase, and an integrase inhibitor. Preferably, the antiviral agent administered to the individual is some combination of effective antiviral therapeutics such as that present in highly active anti-retroviral therapy (“HAART”), a term generally used in the art to refer to a cocktail of 3 or more antiviral drugs, which term includes but is not limited to those combinations of inhibitors of viral protease and reverse transcriptase.

Recombinant, replication-defective adenovirus useful in the methods of the present invention comprise exogenous nucleic acid encoding at least one HIV antigen. The HIV antigen can be any antigen capable of eliciting an immune response in an individual and, most preferably, is derived from an HIV antigen selected from the group consisting of HIV gag, pol, env, nef, rev, tat, vpu, vpr, and vif; or any antigenic/immunogenic portion thereof. The present invention, furthermore, contemplates single and multiple administrations of the recombinant adenovirus expressing the HIV antigen, and accordingly therewith various prime-boost regimens are contemplated for use in the methods of the present invention. In such a scenario, an individual is first administered a priming dose of a viral (or polynucleotide) vehicle comprising nucleic acid encoding an HIV antigen and, following some period of time, administered a boosting dose of a viral (or polynucleotide) vehicle comprising nucleic acid encoding an HIV antigen; provided that either the priming or boosting administration employs an adenoviral vehicle. Preferably, the viral vehicles of the priming and boosting administrations are different in order to evade any host immunity directed against the first delivered vehicle. Selection of the alternate viral vehicle is not critical to the success of the methods disclosed herein. Any viral vehicle capable of delivering the antigen and accomplishing sufficient expression of said antigen such that a cellular-mediated immune response is elicited should be sufficient to prime or boost the adenovirally-mediated administration. The alternative vehicle can be selected from a distinct serotype of adenovirus. Alternatively, the adenoviral administration can be followed or preceded by a viral vehicle of different origin, for instance a pox virus vector, a retrovirus vector, an alpha virus vector, an adeno-associated virus vector, etc. Another embodiment of the present invention employs a prime-boost protocol where adenovirus administration is preceded or followed by polynucleotide administration of nucleic acid encoding an HIV antigen. Yet another embodiment of the present invention employs a prime-boost protocol where adenovirus administration is preceded or followed by delivery of an HIV antigen(s) in the form of a protein/recombinant protein administration.

### BRIEF DESCRIPTION OF THE DRAWINGS

FIGS. 1A-1C illustrate results in the antiretroviral therapy (“ART”)+Vaccine Cohort. (a) Viral loads (RNA copies/mL) are shown for each animal. Arrows indicate the time of initiation of drug therapy (A) and the times for immunization (V). (b) Levels of gag-specific CD8+ T cells (number of gag-specific IFNγ-producing CD8+ cells per 10⁶ lymphocytes) at day 111 (before 1^{st} immunization), 137 (post 1^{st} immunization), 158 (post 2^{nd} immunization), 227 (pre MRKAd6 immunization) and 255 (post MRKAd6 immunization). These were measured by using peptide pools consisting of 20-aa peptide overlapping by 10-aa and encompassing the entire SIVmac239 protein; the values shown here were subtracted for levels in the mock reaction tube. (c) Levels of gag-specific CD4+ T cells (number of gag-specific IFNγ-producing CD8+ cells per 10⁶ lymphocytes) at same assay dates as in (b). The values shown here were also subtracted for levels in the mock reaction tube.

FIGS. 2A-2C illustrate results in the “Vaccine Only” Cohort. (a) Viral loads (RNA copies/mL) are shown for each animal. Arrows indicate the times for immunization (V). (b) Levels of gag-specific CD8+ T cells (number of gag-specific IFNγ-producing CD8+ cells per 10⁶ lymphocytes) at day 111 (before 1^{st} immunization), 137 (post 1^{st} immunization), 158 (post 2^{nd} immunization), 227 (pre MRKAd6 immunization) and 255 (post MRKAd6 immunization). These were measured by using peptide pools consisting of 20-aa peptide overlapping by 10-aa and encompassing the entire SIVmac239 protein; the values shown here were subtracted for levels in the mock reaction tube. (c) Levels of gag-specific CD4+ T cells (number of gag-specific IFNγ-producing CD8+ cells per 10⁶ lymphocytes) at same assay dates as in (b). The values shown here were also subtracted for levels in the mock reaction tube.

FIGS. 3A-3C illustrate results in the “ART Only” Cohort. (a) Viral loads (RNA copies/mL) are shown for each animal. Arrow indicates the time of initiation of drug therapy (A). (b) Levels of gag-specific CD8+ T cells (number of gag-specific IFNγ-producing CD8+ cells per 10⁶ lymphocytes) at day 111, 137, 158, 227 and 255. These were measured by using peptide pools consisting of 20-aa peptide overlapping by 10-aa and encompassing the entire SIVmac239 protein; the values shown here were subtracted for levels in the mock reaction tube. (c) Levels of gag-specific CD4+ T cells (number of gag-specific IFNγ-producing CD8+ cells per 10⁶ lymphocytes) at same assay dates as in (b). The values shown here were also subtracted for levels in the mock reaction tube.

FIGS. 4A-4C illustrate results in the “No Treatment” Control Cohort. (a) Viral loads (RNA copies/mL) are shown for each animal. (b) Levels of gag-specific CD8+ T cells (number of gag-specific IFNγ-producing CD8+ cells per 10⁶ lymphocytes) at day 111, 137, 158, 227 and 255. These were measured by using peptide pools consisting of 20-aa peptide overlapping by 10-aa and encompassing the entire SIVmac239 protein; the values shown here were subtracted for levels in the mock reaction tube. (c) Levels of gag-specific CD4+ T cells (number of gag-specific IFNγ-producing CD8+ cells per 10⁶ lymphocytes) at same assay dates as in (b). The values shown here were also subtracted for levels in the mock reaction tube.

FIGS. 5A-5D illustrate the breadth of gag-specific T cell responses. Positivity to a gag subpool is determined by a response greater than or equal to 50 SFC/10⁶ PBMC in an IFNγ ELISPOT assay. The maximum score is 10. PBMCs were assayed for each animal at day 74 (pre 1^{st} immunization), 158 (post 2^{nd} immunization, and 269 (post 3^{rd} immunization). (a) ART+Vaccine cohort. (b) “Vaccine only” cohort. (c) “ART only” cohort. (d) “No treatment” control cohort.

FIG. 6 illustrates a codon-optimized nucleic acid sequence encoding SIV mac239 gag (SEQ ID NO:1).

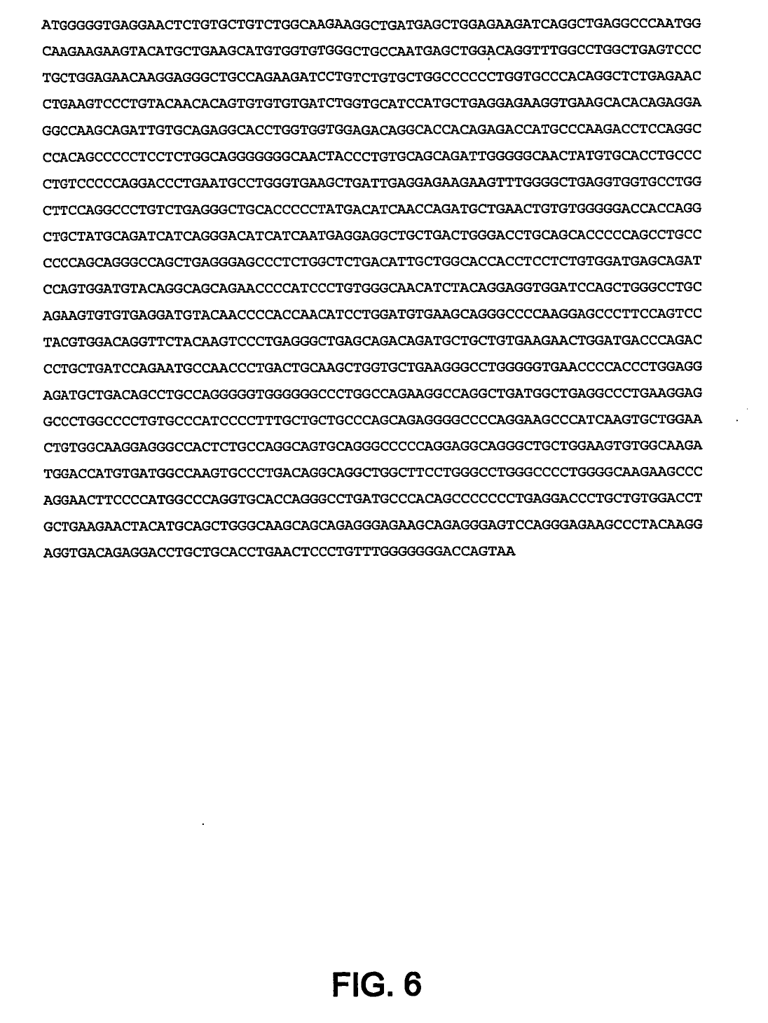

FIG. 7 illustrates a codon-optimized nucleic acid sequence encoding SIV mac251 nef with a G2A mutation (SEQ ID NO:2).

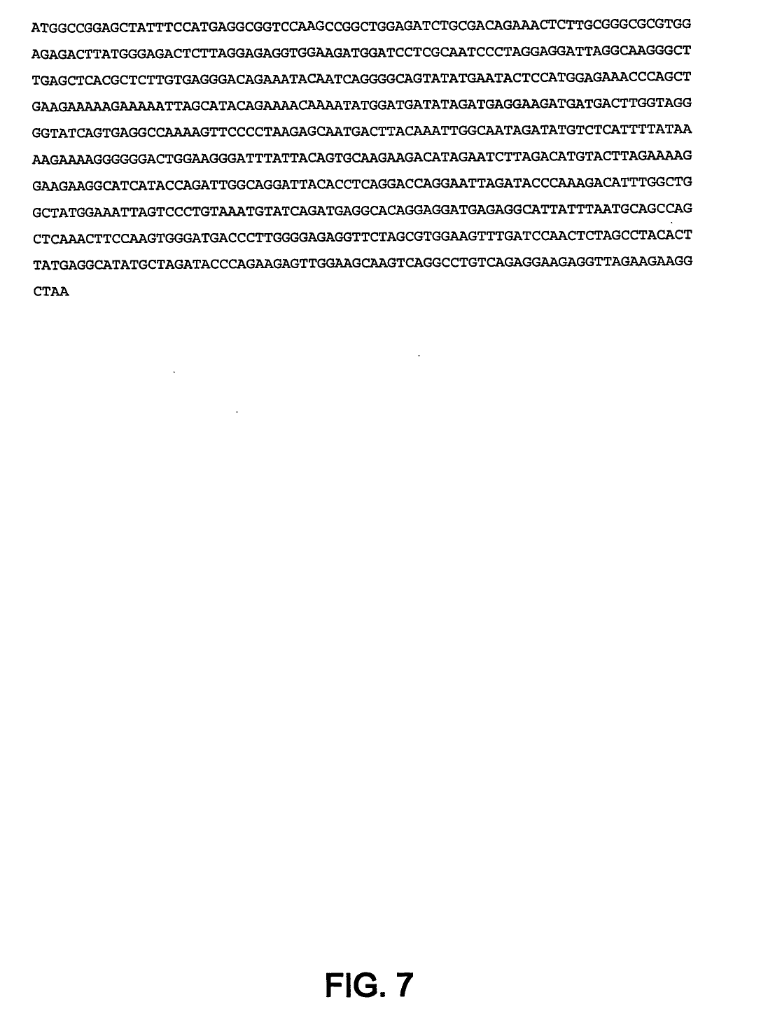

### DETAILED DESCRIPTION OF THE INVENTION

A novel method for eliciting a therapeutic immune response in HIV-infected individuals characterized as having controlled viremia is described. The method comprises administering to an infected individual a recombinant, replication-defective adenovirus comprising exogenous nucleic acid encoding at least one HIV antigen; wherein said individual has experienced, prior to or simultaneous with, the administration, a reduction in HIV viral copy number. The specific cause behind the reduction in viral copy number (i.e., viral load) at the time of immunization is not critical. The reduction can be mediated by an innate ability for the immune system to respond to the presence of the virus; a prior immunization which assists the individual in keeping the viral load at bay; treatment with antiviral agents; or any other reason which perhaps may even remain unascertained. What is important is the finding that immunization of treated individuals in this manner (i.e., with an adenoviral vehicle at this stage of infection) has been found to effectively elicit virus-specific cellular-mediated immune responses in the individuals, as evidenced by a notable increase in virus-specific cytotoxic CD8+ and helper CD4+ T cell responses in treated macaques infected with SIV. The therapeutic immune response that ensues has the capability of effectively maintaining low titers of virus and, thus, offers the prospect of reducing individual dependency on antiviral therapy.

The specific antiviral agent(s) used in the treatment of the infected individual does not bear on the utility of the present methods. The antiviral agent can, for example, be based on/derived from an antibody, a polynucleotide, a polypeptide, a peptide, or a small molecule. Any antiviral agent which effectively reduces viral replication/viral load within an individual should sufficiently prime an individual subject for immunization in accordance with the methods disclosed herein. Antiviral agents antagonize the functioning/life cycle of the virus, and target a protein/function essential to the proper life cycle of the virus; an effect that can be readily determined by an in vivo or in vitro assay. Some representative antiviral agents which target specific viral proteins are protease inhibitors, reverse transcriptase inhibitors (including nucleoside analogs; non-nucleoside reverse transcriptase inhibitors; and nucleotide analogs), and integrase inhibitors. Protease inhibitors include, for example, indinavir/CRIXIVAN®; ritonavir/NORVIR®; saquinavir/FORTOVASE®; nelfinavir/VIRACEPT®; amprenavir/AGENERASE®; lopinavir and ritonavir/KALETRA®. Reverse transcriptase inhibitors include, for example, (1) nucleoside analogs, e.g., zidovudine/RETROVIR® (AZT); didanosine/VIDEX® (ddI); zalcitabine/HIVID® (ddC); stavudine/ZERIT® (d4T); lamivudine/EPIVIR® (3TC); abacavir/ZIAGEN®D (ABC); (2) non-nucleoside reverse transcriptase inhibitors, e.g., nevirapine/VIRAMUNE® (NVP); delavirdine/RESCRIPTOR® (DLV); efavirenz/SUSTIVA® (EFV); and (3) nucleotide analogs, e.g., tenofovir DF/VIREAD® (TDF). Integrase inhibitors include, for example, the molecules disclosed in U.S. Application Publication No. US2003/0055071, published Mar. 20, 2003; and International Application WO 03/035077. The antiviral agents, as indicated, can target as well a function of the virus/viral proteins, such as, for instance the interaction of regulatory proteins tat or rev with the trans-activation response region (“TAR”) or the rev-responsive element (“RRE”), respectively.

The present invention contemplates as well the immunization of individuals that have been treated with a combination of antiviral agents. For example, antiviral agents may be administered in combination with effective amounts of the HIV/AIDS antivirals, immunomodulators, anti-infectives, or vaccines useful for treating HIV infection or AIDS, including but not limited to those in the following table:

| | |
|---|---|
| Drug Name | Indication (Activity) |

ANTIVIRALS

| | | |
|---|---|---|
| | Manufacturer | |
| | (Tradename and/or | |
| | Location) | |
| Abacavir | Glaxo Welcome | HIV infection, AIDS, ARC |
| GW 1592 | (ZIAGEN ®) | (nucleoside reverse |
| 1592U89 | | transcriptase inhibitor) |
| abacavir + lamivudine + zidovudine | GlaxoSmithKline | HIV infection, AIDS, ARC |
| | (TRIZIVIR ®) | (nucleoside reverse |
| | | transcriptase inhibitors) |
| acemannan | Carrington Labs | ARC |
| | (Irving, TX) | |
| ACH 126443 | Achillion Pharm. | HIV infections, AIDS, ARC |
| | | (nucleoside reverse |
| | | transcriptase inhibitor) |
| acyclovir | Burroughs Wellcome | HIV infection, AIDS, ARC, |
| | | in combination with AZT |
| AD-439 | Tanox Biosystems | HIV infection, AIDS, ARC |
| AD-519 | Tanox Biosystems | HIV infection, AIDS, ARC |
| adefovir dipivoxil | Gilead | HIV infection, AIDS, ARC |
| GS 840 | | (reverse transcriptase |
| | | inhibitor) |
| AL-721 | Ethigen | ARC, PGL, HIV positive, |
| | (Los Angeles, CA) | AIDS |
| alpha interferon | GlaxoSmithKline | Kaposi's sarcoma, HIV, in |
| | | combination w/Retrovir |
| AMD3100 | AnorMed | HIV infection, AIDS, |
| | | ARC |
| | | (CXCR4 antagonist) |
| Amprenavir | GlaxoSmithKline | HIV infection, AIDS, |
| 141 W94 | (AGENERASE ®) | ARC (protease inhibitor) |
| GW 141 | | |
| VX478 (Vertex) | | |
| Ansamycin | Adria Laboratories | ARC |
| LM 427 | (Dublin, OH) | |
| | Erbamont | |
| | (Stamford, CT) | |
| antibody which neutralizes | Advanced Biotherapy | AIDS, ARC |
| pH labile alpha aberrant | Concepts (Rockville, | |
| interferon | MD) | |
| AR177 | Aronex Pharm | HIV infection, AIDS, ARC |
| atazanavir (BMS 232632) | Bristol-Myers Squibb | HIV infection, AIDS, ARC |
| | (REYATAZ ™) | (protease inhibitor) |
| beta-fluoro-ddA | Nat'l Cancer Institute | AIDS-associated diseases |
| BMS-232623 | Bristol-Myers Squibb/ | HIV infection, AIDS, |
| (CGP-73547) | Novartis | ARC |
| | | (protease inhibitor) |
| BMS-234475 | Bristol-Myers Squibb/ | HIV infection, AIDS, |
| (CGP-61755) | Novartis | ARC (protease inhibitor) |
| Capravirine | Pfizer | HIV infection, AIDS, |
| (AG-1549, S-1153) | | ARC (non-nucleoside |
| | | reverse transcriptase |
| | | inhibitor) |
| CI-1012 | Warner-Lambert | HIV-1 infection |
| Cidofovir | Gilead Science | CMV retinitis, herpes, |
| | | papillomavirus |
| curdlan sulfate | AJI Pharma USA | HIV infection |
| cytomegalovirus immune | MedImmune | CMV retinitis |
| globin | | |
| cytovene | Syntex | sight threatening CMV |
| ganciclovir | | peripheral CMV |
| | | retinitis |
| Delavirdine | Pharmacia-Upjohn | HIV infection, AIDS, |
| | (RESCRIPTOR ®) | ARC (non-nucleoside |
| | | reverse transcriptase |
| | | inhibitor) |
| dextran Sulfate | Ueno Fine Chem. Ind. | AIDS, ARC, HIV |
| | Ltd. (Osaka, Japan) | positive asymptomatic |
| DdC | Hoffman-La Roche | HIV infection, AIDS, ARC |
| (zalcitabine, | (HIVID ®) | (nuclesodie reverse |
| dideoxycytidine) | | transcriptase inhibitor) |
| ddI | Bristol-Myers Squibb | HIV infection, AIDS, ARC; |
| (didanosine, | (VIDEX ®) | combination with AZT/d4T |
| dideoxyinosine) | | (nucleoside reverse |
| | | transcriptase inhibitor) |
| DPC 681 & DPC 684 | DuPont | HIV infection, AIDS, ARC |
| | | (protease inhibitors) |
| DPC 961 & DPC 083 | Bristol-Myers Squibb | HIV infection AIDS, ARC |
| | (from DuPont Pharma) | (non-nucleoside reverse |
| | | transcriptase inhibitors) |
| EL10 | Elan Corp, PLC | HIV infection |
| | (Gainesville, GA) | |
| efavirenz | Bristol-Myers Squibb | HIV infection, AIDS, |
| (DMP 266) | (SUSTIVA ®) | ARC (non-nucleoside RT |
| | Merck (STOCRIN ®) | inhibitor) |
| Famciclovir | Novartis | herpes zoster, herpes |
| | (FAMVIR ®) | simplex |
| Emtricitabine | Gilead (from Triangle | HIV infection, AIDS, ARC |
| FTC | Pharmaceuticals) | (nucleoside reverse |
| | (COVIRACIL ®) | transcriptase inhibitor) |
| | Emory University | |
| Emvirine | Gilead (from Triangle | HIV infection, AIDS, ARC |
| | Pharmaceuticals) | (non-nucleoside reverse |
| | (COACTINON ®) | transcriptase inhibitor) |
| Enfuvirtide | Trimeris & Roche | HIV infection, AIDS, ARC |
| T-20 | (FUZEON ®) | (fusion inhibitor) |
| HBY097 | Hoechst Marion Roussel | HIV infection, AIDS, ARC |
| | | (non-nucleoside reverse |
| | | transcriptase inhibitor) |
| Fosamprenavir | Glaxo Smith Kline | HIV infection, AIDS, ARC |
| | | (prodrug of amprenavir) |
| Hypericin | VIMRx Pharm. | HIV infection, AIDS, ARC |
| recombinant human | Triton Biosciences | AIDS, Kaposi's sarcoma, |
| interferon beta | (Almeda, CA) | ARC |
| interferon alfa-n3 | Interferon Sciences | ARC, AIDS |
| Indinavir | Merck (CRIXIVAN ®) | HIV infection, AIDS, ARC, |
| | | asymptomatic HIV positive, |
| | | (protease inhibitor) |
| ISIS 2922 | ISIS Pharmaceuticals | CMV retinitis |
| JE2147/AG1776 | Agouron | HIV infection, AIDS, ARC |
| | | (protease inhibitor) |
| KNI-272 | Nat'l Cancer Institute | HIV-assoc. diseases |
| lamivudine, 3TC | GlaxoSmithKline | HIV infection, AIDS, |
| | (EPIVIR ®) | ARC (nucleoside reverse |
| | | transcriptase inhibitor) |
| lamivudine + zidovudine | GlaxoSmithKline | HIV infection, AIDS, |
| | (COMBIVIR ®) | ARC (nucleoside reverse |
| | | transcriptase inhibitor) |
| Lobucavir | Bristol-Myers Squibb | CMV infection |
| lopinavir (ABT-378) | Abbott | HIV infection, AIDS, ARC |
| | | (protease inhibitor) |
| lopinavir + ritonavir | Abbott (KALETRA ®) | HIV infection, AIDS, ARC |
| (ABT-378/r) | | (protease inhibitor) |
| mozenavir | AVID (Camden, NJ) | HIV infection, AIDS, ARC |
| (DMP-450) | | (protease inhibitor) |
| Nelfinavir | Agouron | HIV infection, AIDS, |
| | (VIRACEPT ®) | ARC (protease inhibitor) |
| Nevirapine | Boeheringer | HIV infection, AIDS, |
| | Ingleheim | ARC (non-nucleoside |
| | (VIRAMUNE ®) | reverse transcriptase |
| | | inhibitor) |
| Novapren | Novaferon Labs, Inc. | HIV inhibitor |
| | (Akron, OH) | |
| peptide T | Peninsula Labs | AIDS |
| octapeptide | (Belmont, CA) | |
| sequence | | |
| PRO 140 | Progenics | HIV infection, AIDS, ARC |
| | | (CCR5 co-receptor inhibitor) |
| PRO 542 | Progenics | HIV infection, AIDS, ARC |
| | | (attachment inhibitor) |
| Trisodium | Astra Pharm. Products, | CMV retinitis, HIV infection, |
| phosphonoformate | Inc | other CMV infections |
| PNU-140690 | Pharmacia Upjohn | HIV infection, AIDS, ARC |
| | | (protease inhibitor) |
| Probucol | Vyrex | HIV infection, AIDS |
| RBC-CD4 | Sheffield Med. Tech | HIV infection, AIDS, |
| | (Houston TX) | ARC |
| Ritonavir | Abbott (NORVIR ®) | HIV infection, AIDS, |
| (ABT-538) | | ARC (protease inhibitor) |
| Saquinavir | Hoffmann-LaRoche | HIV infection, AIDS, |
| | (FORTOVASE ®) | ARC (protease inhibitor) |
| stavudine; d4T | Bristol-Myers Squibb | HIV infection, AIDS, ARC |
| didehydrodeoxythymidine | (ZERIT ®) | (nucleoside reverse |
| | | transcriptase inhibitor) |
| T-1249 | Trimeris | HIV infection, AIDS, ARC |
| | | (fusion inhibitor) |
| TAK-779 | Takeda | HIV infection, AIDS, ARC |
| | | (injectable CCR5 receptor |
| | | antagonist) |
| Tenofovir | Gilead (VIREAD ®) | HIV infection, AIDS, ARC |
| | | (nucleotide reverse |
| | | transcriptase inhibitor) |
| tipranavir (PNU-140690) | Boehringer Ingelheim | HIV infection, AIDS, ARC |
| | | (protease inhibitor) |
| TMC-120 & TMC-125 | Tibotec | HIV infections, AIDS, ARC |
| | | (non-nucleoside reverse |
| | | transcriptase inhibitors) |
| TMC-126 | Tibotec | HIV infection, AIDS, ARC |
| | | (protease inhibitor) |
| Valaciclovir | GlaxoSmithKline | genital HSV & CMV |
| | | infections |
| Virazole | Viratek/ICN (Costa | asymptomatic HIV positive, |
| ribavirin | Mesa, CA) | LAS, ARC |
| zidovudine; AZT | GlaxoSmithKline | HIV infection, AIDS, ARC, |
| | (RETROVIR ®) | Kaposi's sarcoma in |
| | | combination with other |
| | | therapies (nucleoside reverse |
| | | transcriptase inhibitor) |

IMMUNO-MODULATORS

| | | |
|---|---|---|
| | Manufacturer | |
| AS-101 | Wyeth-Ayerst | AIDS |
| Bropirimine | Pharmacia Upjohn | advanced AIDS |
| Acemannan | Carrington Labs, Inc. | AIDS, ARC |
| | (Irving, TX) | |
| CL246,738 | American Cyanamid | AIDS, Kaposi's sarcoma |
| | Lederle Labs | |
| EL10 | Elan Corp, PLC | HIV infection |
| | (Gainesville, GA) | |
| FP-21399 | Fuki ImmunoPharm | blocks HIV fusion with |
| | | CD4+ cells |
| Gamma Interferon | Genentech | ARC, in combination w/TNF |
| | | (tumor necrosis factor) |
| Granulocyte Macrophage | Genetics Institute | AIDS |
| Colony Stimulating Factor | Sandoz | |
| Granulocyte Macrophage | Hoeschst-Roussel | AIDS |
| Colony Stimulating Factor | Immunex | |
| Granulocyte Macrophage | Schering-Plough | AIDS, combination w/AZT |
| Colony Stimulating Factor | | |
| HIV Core Particle | Rorer | seropositive HIV |
| Immunostimulant | | |
| IL-2 | Cetus | AIDS, in combination |
| Interleukin-2 | | w/AZT |
| IL-2 | Hoffman-La Roche | AIDS, ARC, HIV, in |
| Interleukin-2 | Immunex | combination w/AZT |
| IL-2 | Chiron | AIDS, increase in CD4 cell |
| Interleukin-2 (aldeslukin) | | counts |
| Immune Globulin | Cutter Biological | pediatric AIDS, in |
| ntravenous (human) | (Berkeley, CA) | combination w/AZT |
| IMREG-1 | Imreg (New Orleans, | AIDS, Kaposi's sarcoma, |
| | LA) | ARC, PGL |
| IMREG-2 | Imreg (New Orleans, | AIDS, Kaposi's sarcoma, |
| | LA) | ARC, PGL |
| Imuthiol Diethyl Dithio | Merieux Institute | AIDS, ARC |
| Carbamate | | |
| Alpha-2 Interferon | Schering Plough | Kaposi's sarcoma w/AZT, |
| | | AIDS |
| Methionine-Enkephalin | TNI Pharmaceutical | AIDS, ARC |
| | (Chicago, IL) | |
| MTP-PE | Ciba-Geigy Corp. | Kaposi's sarcoma |
| Muramyl-Tripeptide | | |
| Granulocyte Colony | Amgen | AIDS, in combination |
| Stimulating Factor | | w/AZT |
| Remune | Immune Response Corp. | immunotherapeutic |
| rCD4 Recombinant | Genentech | AIDS, ARC |
| Soluble Human CD4 | | |
| rCD4-IgG hybrids | | AIDS, ARC |
| Recombinant Soluble | Biogen | AIDS, ARC |
| Human CD4 | | |
| Interferon Alfa 2a | Hoffman-La Roche | Kaposi's sarcoma, AIDS, |
| | | ARC, in combination w/AZT |
| SK&F106528 | Smith Kline | HIV infection |
| Soluble T4 | | |
| Thymopentin | Immunobiology | HIV infection |
| | Research Institute | |
| Tumor Necrosis Factor; | Genentech | ARC, in combination |
| TNF | | w/gamma Interferon |
| Etanercept | Immunex Corp | rheumatoid arthritis |
| | (ENBREL ®) | |
| Infliximab | Centocor | rheumatoid arthritis and |
| | (REMICADE ®) | Crohn's disease |

ANTI-INFECTIVES

| | | |
|---|---|---|
| | Manufacturer | |
| Clindamycin with | Pharmacia Upjohn | PCP |
| Primaquine | | |
| Fluconazole | Pfizer | cryptococcal meningitis, |
| | | candidiasis |
| Pastille Nystatin Pastille | Squibb Corp. | prevention of oral candidiasis |
| Ornidyl Eflornithine | Merrell Dow | PCP |
| Pentamidine Isethionate | LyphoMed | PCP treatment |
| (IM & IV) | (Rosemont, IL) | |
| Trimethoprim | | antibacterial |
| Trimethoprim/sulfa | | antibacterial |
| Piritrexim | Burroughs Wellcome | PCP treatment |
| Pentamidine isethionate | Fisons Corporation | PCP prophylaxis |
| for inhalation | | |
| Spiramycin | Rhone-Poulenc | cryptosporidia diarrhea |
| Intraconazole-R51211 | Janssen Pharm. | histoplasmosis; cryptococcal |
| | | meningitis |
| Trimetrexate | Warner-Lambert | PCP |

OTHER

| | | |
|---|---|---|
| | Manufacturer | |
| Daunorubicin | NeXstar, Sequus | Karposi's sarcoma |
| Recombinant Human | Ortho Pharm. Corp. | severe anemia assoc. with |
| Erythropoietin | | AZT therapy |
| Recombinant Human | Serono | AIDS-related wasting, |
| Growth Hormone | | cachexia |
| Leukotriene B4 Receptor | — | HIV infection |
| Antagonist | | |
| Megestrol Acetate | Bristol-Myers Squibb | treatment of anorexia assoc. |
| | | w/AIDS |
| Soluble CD4 Protein and | — | HIV infection |
| Derivatives | | |
| Testosterone | Alza, Smith Kline | AIDS-related wasting |
| Total Enteral Nutrition | Norwich Eaton | diarrhea and malabsorption, |
| | Pharmaceuticals | related to AIDS |

It will be understood that the scope of combinations of antiviral agents that can be used to reduce viral load prior to immunization in accordance with the methods disclosed herein is not limited to the above Table, but includes in principle any combination with any pharmaceutical composition useful for the treatment of HIV infection or AIDS. When employed as a therapeutic for the treatment of HIV/AIDS, antivirals and other agents are typically employed in their conventional dosage ranges and regimens as reported in the art, including the dosages described in the Physicians' Desk Reference, 54^{th} edition, Medical Economics Company, 2000.

Antiviral interference with the viral life cycle and consequent effect on viral load can be measured, inter alia, by analyzing the number of viral copies present within the individual before, during and/or after treatment. This measurement can be used as an indicator as to the success/failure of any specific antiviral treatment regimen and forms the basis for predicting an individual's diagnosis or risk of clinical progression. Specific individuals can generate a resistance to certain antivirals and, thus, it is important to monitor the degree of success of any particular antiviral treatment regimen. Viral load is a measurement of the amount of virus/virally infected cells in the cells, blood plasma or tissues of a patient. While there are no absolute numbers associated with disease progression, certain levels of virus in the plasma have been classified as telling of an individual's infection status. A reduction in plasma HIV RNA levels has been associated with increased survival and a reduced likelihood of progressing to disease. Consequently, it appears that the higher the levels of virus, the more rapid the onset of disease. Very high levels of virus are said to be present where there is approximately 100,000 copies or more of HIV RNA per ml of plasma; high levels of virus are said to be present when there are approximately 30,000-50,000 copies of HIV RNA per ml of plasma; and low levels of virus are said to be present when there are approximately 5,000-10,000 copies of HIV RNA per ml of plasma; Carpenter et al., 1996 JAMA 276:147-154. There are several means available to make a determination as to viral load, whether direct or indirect, by assays performed on patient blood cells, tissue, serum and plasma; see, e.g., “Report of the NIH to Define Principles of Therapy of HIV Infection”, Apr. 24, 1998 issue of Morbidity & Mortality Weekly Reports, 47 (No. RR-5); revised Jun. 17, 1998; Voldberding & Jacobson, 1992 AIDS Clinical Review (Marcel Dekker, Inc., N.Y.). Available techniques to measure viral RNA or DNA include, but are not limited to, the following: polymerase chain reaction (“PCR”) amplification techniques (e.g., WO 94/20640; AMPLICOR®D; Sambrook et al., 1989 Molecular Cloning: A Laboratory Manual, 2d Edition (Cold Spring Harbor press, Cold Spring Harbor, N.Y.; Ausubel et al., 1994 Current Protocols in Molecular Biology (Green Publishing Associates and John Wiley & Sons, New York, N.Y.; and PCR Protocols, 1991 (Cold Spring Harbor, N.Y.); branched DNA (“bDNA”) tests (e.g., WO 92/02526; U.S. Pat. No. 5,451,503; U.S. Pat. No. 4,775,619; QUANIPLEX®; VERSANT®); standard hybridization (including the use of probes in hybridization, see, e.g., EP 617,132); and antibody detection methods. Viral load should be measured before treatment with antiviral agents. Effective treatment with antiviral drugs has been reported to decrease viral load by 90% or more within 8 weeks, and thereafter continue to decrease viral load through to undetectable levels within 6 months. Preferably, the antiviral agents administered prior to vaccination in accordance with the methods of the present invention effect a decrease in viral load that brings the viral load to ⅓ or better of what it was at steady state levels of virus; and, more preferably, to “undetectable” levels (a term defined by the technology available at the time and the specific technology employed).

Applicants have identified a correlation between the presence/absence of controlled viremia and the benefit of an immunization protocol employing recombinant, replication-defective adenovirus in the delivery of nucleic acid encoding an HIV antigen. Accordingly, the instant invention is based on the immunization of HIV-infected individuals within whom viral load is controlled (i.e., viral load levels having been reduced from that existing at some prior time point). An embodiment of the instant invention, thus, comprises the therapeutic immunization of HIV-infected individuals following or simultaneous with controlled viremia; controlled viremia being defined as a reduction in viral load, be that from a predisposed (immunized)/innate immune response, treatment with antiviral agents, or other. Adenovirus has been identified as capable of effecting a virus-specific cellular-mediated immune response in infected, immunized subjects.

Adenoviruses are nonenveloped, icosahedral viruses that have been identified in several avian and mammalian hosts; Home et al., 1959 J. Mol. Biol. 1:84-86; Horwitz, 1990 In Virology, eds. B. N. Fields and D. M. Knipe, pps. 1679-1721. The first human adenoviruses (Ads) were isolated over four decades ago. Since then, over 100 distinct adenoviral serotypes have been isolated which infect various mammalian species, 51 of which are of human origin; Straus, 1984, In The Adenoviruses, ed. H. Ginsberg, pps. 451498, New York: Plenus Press; Hierholzer et al., 1988 J. Infect. Dis. 158:804-813; Schnurr and Dondero, 1993, Intervirology; 36:79-83; Jong et al., 1999 J Clin Microbiol., 37:3940-5. The human serotypes have been categorized into six subgenera (A-F) based on a number of biological, chemical, immunological and structural criteria which include hemagglutination properties of rat and rhesus monkey erythrocytes, DNA homology, restriction enzyme cleavage patterns, percentage G+C content and oncogenicity; Straus, supra; Horwitz, supra.

The adenovirus genome is very well characterized. It consists of a linear double-stranded DNA molecule of approximately 36,000 base pairs, and despite the existence of several distinct serotypes, there is some general conservation in the overall organization of the adenoviral genome with specific functions being similarly positioned.

Adenovirus has been a very attractive target for delivery of exogenous genes. The biology of adenoviruses is very well understood. Adenovirus has not been found to be associated with severe human pathology in immuno-competent individuals. The virus is extremely efficient in introducing its DNA into the host cell and is able to infect a wide variety of cells. Furthermore, the virus can be produced at high virus titers in large quantities. In addition, the virus can be rendered replication defective by deletion/modification of the essential early-region 1 (E1) of the viral genome, rendering the virus devoid (or essentially devoid) of E1 activity and, thus, incapable of replication in the intended host/vaccinee; see, e.g., Brody et al, 1994 Ann N Y Acad Sci., 716:90-101. Deletion of adenoviral genes other than E1 (e.g., in E3, E2 and/or E4) have created adenoviral vectors with greater capacity for exogenous gene inclusion, which adenoviral vectors have proven to be effective gene delivery vehicles as well. Accordingly, such vectors are suitable for use in the methods of the present invention. For many of the above reasons, adenovirus vectors have been used extensively as gene transfer vectors for vaccine and gene therapy purposes.

Presently, two well-characterized adenovirus serotypes from subgroup C, Ad5 and Ad2, are the most widely used gene delivery vectors. Adenovirus serotype 5 has been found to be a very effective adenovirus vehicle for purposes of effectuating expression of exogenous genetic material. The wildtype adenovirus serotype 5 sequence is known and described in the art; see, Chroboczek et al., 1992 J. Virology 186:280, which is hereby incorporated by reference. Accordingly, a particular embodiment of the present invention is an immunization scheme employing an adenovirus vehicle based on the wildtype adenovirus serotype 5 sequence in the priming or boosting administration; a virus of which is on deposit with the American Type Culture Collection (“ATCC”) under ATCC Deposit No. VR-5. A further embodiment is an immunization scheme in accordance with the present invention wherein the adenoviral vector employed (whether Ad5, Ad6 or other) is as described in WO 02/22080; which is hereby incorporated by reference. Said vectors are at least partially deleted in E1 and comprise the several adenoviral packaging repeats (i.e., the E1 deletion does not start until approximately base pairs 450458 corresponding to a wildtype Ad5 sequence). These properties have been found to greatly enhance growth characteristics/properties of the virus.

While the present invention can effectively be carried out using adenovirus serotypes 2, 5 or 6 (ATCC Deposit No. VR-6; see, e.g, WO 03/31588, published Apr. 17, 2003), it is contemplated herein that alternative and distinct human and non-human adenovirus can be used in the disclosed methods either in a single administration regimen or in combined administration with another viral vehicle, or polynucleotide/protein administration. One of skill in the art can readily identify alternative and distinct adenovirus serotypes (e.g., the various serotypes found in subgenera A-F discussed above; including but not limited to Ad7; Ad35 (see, e.g., EP1054064); Ad24; Ad34; etc.) and non-human serotypes (including but not limited to primate adenovirus (see, e.g., Fitzgerald et al., 2003 J. Immunol. 170(3):1416-1422; Xiang et al., 2002 J. Virol. 76(6):2667-2675)); and incorporate same in the methods disclosed herein. Alternate Ad serotypes are desirable in that they possess the ability to evade neutralizing antibodies to adenoviral serotypes more prevalent in the general population. Alternate serotypes, as well, possess alternate tropisms which may lead to the elicitation of superior immune responses when used for vaccine or gene therapy purposes.

Adenoviral vectors suitable for use in the methods of the instant invention can be constructed using known techniques, such as those reviewed in Hitt et al., 1997 “Human Adenovirus Vectors for Gene Transfer into Mammalian Cells” Advances in Pharmacology 40:137-206, which is hereby incorporated by reference. Often, a plasmid or shuttle vector containing the heterologous nucleic acid of interest is generated which comprises sequence homologous to the specific adenovirus of interest. The shuttle vector and viral DNA or second plasmid containing the cloned viral DNA are then co-transfected into a host cell where homologous recombination occurs resulting in the incorporation of heterologous nucleic acid into the viral nucleic acid. Preferred shuttle vectors and cloned viral genomes contain adenoviral and plasmid portions. For shuttle vectors used in the construction of replication-defective vectors, the adenoviral portion typically contains non-functional or deleted E1 and E3 regions and the gene expression cassette, flanked by convenient restriction sites. The plasmid portion of the shuttle vector typically contains an antibiotic resistance marker under the transcriptional control of a prokaryotic promoter. Ampicillin resistance genes, neomycin resistance genes and other pharmaceutically acceptable antibiotic resistance markers may be used. To aid in high level production of nucleic acid by fermentation in prokaryotic organisms, it is advantageous for the shuttle vector to contain a prokaryotic origin of replication and be of high copy number. A number of commercially available prokaryotic cloning vectors provide these benefits. Non-essential DNA sequences are, preferably removed. It is also preferable that the vectors not be able to replicate in eukaryotic cells. This minimizes the risk of integration of nucleic acid vaccine sequences into the recipients' genome. Tissue-specific promoters or enhancers may be used whenever it is desirable to limit expression of the nucleic acid to a particular tissue type. Homologous recombination of the shuttle vector and wild-type adenovirus viral DNA (Ad backbone vector) results in the generation of adenoviral pre-plasmids. Upon linearization, the pre-plasmids are capable of replication in PER.C6® cells or alternative E1-complementing cell lines. Infected cells and media can then be harvested once viral replication is complete. The harvested material can then be purified, formulated, and stored prior to host administration.

E1-complementing cell lines used for the propagation and rescue of recombinant adenovirus should provide elements essential for the virus to replicate, whether the elements are encoded in the cell's genetic material or provided in trans. It is, furthermore, preferable that the E1-complementing cell line and the vector not contain overlapping elements which could enable homologous recombination between the DNA of the vector and the DNA of the cell line potentially leading to replication competent virus (or replication competent adenovirus (“RCA”)). Typically, E1-complementing cells are human cells derived from the retina or kidney, although any cell line capable of expressing the appropriate E1 and any other critical deleted region(s) can be utilized to generate adenovirus suitable for use in the methods of the present invention. Embryonal cells such as amniocytes have been shown to be particularly suited for the generation of E1 complementing cell lines. Several cell lines are available and include but are not limited to the known cell lines PER.C6® (ECACC deposit number 96022940), 911, 293, and E1 A549. PER.C6®D cell lines are described in WO 97/00326 (published Jan. 3, 1997) and issued U.S. Pat. No. 6,033,908, both of which are hereby incorporated by reference. PER.C6® is a primary human retinoblast cell line transduced with an E1 gene segment that complements the production of replication deficient (FG) adenovirus, but is designed to prevent generation of replication competent adenovirus by homologous recombination. 293 cells are described in Graham et al., 1977 J. Gen. Virol 36:59-72, which is hereby incorporated by reference. For the propagation and rescue of non-group C adenoviral vectors, a cell line expressing an E1 region which is complementary to the E1 region deleted in the virus being propagated can be utilized. Alternatively, a cell line expressing regions of E1 and E4 derived from the same serotype can be employed; see, e.g., U.S. Pat. No. 6,270,996. Another alternative would be to propagate non-group C adenovirus in available E1-expressing cell lines (e.g., PER.C6®, A549 or 293). This latter method involves the incorporation of a critical E4 region into the adenovirus to be propagated. The critical E4 region is native to a virus of the same or highly similar serotype as that of the E1 gene product(s) (particularly the E1B 55K region) of the complementing cell line, and comprises, in the least, nucleic acid encoding E4 Orf6. One of skill in the art can readily appreciate and carry out numerous other methods suitable for the production of recombinant, replication-defective adenovirus suitable for use in the methods of the present invention.

Recombinant adenovirus suitable for use in the instant invention comprise exogenous nucleic acid encoding an HIV antigen or an immunologically relevant modification thereof. HIV antigens of interest include, but are not limited to, the major structural proteins of HIV such as Gag, Pol, and Env (including gp160, gp120 and gp41); regulatory proteins (e.g., Tat and Rev); and accessory proteins (e.g., Vpu, Vpr, Vif and Nef); immunologically relevant modifications/derivatives of the foregoing, and immunogenic portions thereof. The invention contemplates as well the various codon-optimized forms of nucleic acid encoding HIV antigens, including codon-optimized HIV gag (including but by no means limited to p55 versions of codon-optimized full length (“FL”) Gag and tPA-Gag fusion proteins), HIV pol, HIV nef, HIV env, HIV tat, HIV rev, and selected modifications of immunological relevance. Specific embodiments employ the recombinant, replication defective adenovirus comprising gag, pol, and nef antigens disclosed in WO 02/22080; which is hereby incorporated by reference. A codon-optimized HIV-1 gag gene is disclosed in WO 02/22080. Codon-optimized HIV-1 env genes are disclosed in PCT International Applications WO 97/31115 and WO 97/48370. Codon-optimized HIV-1 pol genes are disclosed in U.S. application Ser. No. 09/745,221, filed Dec. 21, 2000 and WO 01/45748. Codon-optimized HIV-1 nef genes are disclosed in U.S. application Ser. No. 09/738,782, filed Dec. 15, 2000 and WO 01/43693. It is well within the purview of the skilled artisan to choose an appropriate nucleotide sequence including but not limited to those cited above which encodes a specific HIV antigen, or immunologically relevant portion or modification/derivative thereof. “Immunologically relevant” or “antigenic” as defined herein means (1) with regard to a viral antigen, that the protein is capable, upon administration, of eliciting a measurable immune response within an individual sufficient to retard the propagation and/or spread of the virus and/or to reduce the viral load present within the individual; or (2) with regards to a nucleotide sequence, that the sequence is capable of encoding for a protein capable of the above.

In addition to a single protein or antigen of interest being delivered by the recombinant, replication-defective adenovirus, two or more proteins or antigens can be delivered either via separate vehicles or delivered via the same vehicle. Multiple genes/functional equivalents may be ligated into a proper shuttle plasmid for generation of a pre-adenoviral plasmid comprising multiple open reading frames. Open reading frames for the multiple genes/functional equivalents can be operatively linked to distinct promoters and transcription termination sequences. In other embodiments, the open reading frames may be operatively linked to a single promoter, with the open reading frames operatively linked by an internal ribosome entry sequence (IRES; as disclosed in WO 95/24485), or suitable alternative allowing for transcription of the multiple open reading frames to run off of a single promoter. In certain embodiments, the open reading frames may be fused together by stepwise PCR or suitable alternative methodology for fusing together two open reading frames. An example of a gag-pol fusion construct and various other combined modality administration regimens suitable for use in the present invention are disclosed in WO 02/22080; which is hereby incorporated by reference. It is well within the purview of one of skill in the art to arrive at and effectively utilize fusion constructs constructed from diverse combinations of the several art-recognized HIV antigens, including but not limited to gag-pol-nef fusions. In all constructs of use herein, due consideration must be given to the effective packaging limitations of the viral vehicle. Adenovirus type 5, for instance, has been shown to exhibit an upper cloning capacity limit of approximately 105% of the wildtype Ad5 sequence.

The exogenous nucleic acid may be derived from any HIV strain, including but not limited to HIV-1 and HIV-2, strains A, B, C, D, E, F, G, H, I, O, IIIB, LAV, SF2, CM235, and US4; see, e.g., Myers et al, eds. “Human Retroviruses and AIDS: 1995 (Los Alamos National Laboratory, Los Alamos N. Mex. 87545); hereby incorporated by reference. Another HIV strain suitable for use in the methods disclosed herein is HIV-1 strain CAM-1; Myers et al, eds. “Human Retroviruses and AIDS: 1995, IIA3-IIA19, which is hereby incorporated by reference. This gene closely resembles the consensus amino acid sequence for the clade B (North American/European) sequence. HIV gene sequence(s) may be based on various clades of HIV-1; specific examples of which are Clades B and C. Sequences for genes of many HIV strains are publicly available from GenBank and primary, field isolates of HIV are available from the National Institute of Allergy and Infectious Diseases (NIAID) which has contracted with Quality Biological (Gaithersburg, Md.) to make these strains available. Strains are also available from the World Health Organization (WHO), Geneva Switzerland.

The exogenous nucleic acid can be DNA and/or RNA, and can be double or single stranded. The nucleic acid can be inserted in an E1 parallel (transcribed 5′ to 3′) or anti-parallel (transcribed in a 3′ to 5′ direction relative to the vector backbone) orientation. The nucleic acid can be codon-optimized for expression in the desired host (e.g., a mammalian host). The heterologous nucleic acid can be in the form of an expression cassette. A gene expression cassette will typically contain (a) nucleic-acid encoding a protein or antigen of interest; (b) a heterologous promoter operatively linked to the nucleic acid encoding the protein; and (c) a transcription termination signal. In specific embodiments, the heterologous promoter is recognized by a eukaryotic RNA polymerase. One example of a promoter suitable for use in the present invention is the immediate early human cytomegalovirus promoter (Chapman et al., 1991 Nucl. Acids Res. 19:3979-3986). Further examples of promoters that can be used in the present invention are the strong immunoglobulin promoter, the EF1 alpha promoter, the murine CMV promoter, the Rous Sarcoma Virus promoter, the SV40 early/late promoters and the beta actin promoter, albeit those of skill in the art can appreciate that any promoter capable of effecting expression in the intended host can be used in accordance with the methods of the present invention. The promoter may comprise a regulatable sequence such as the Tet operator sequence. Sequences such as these that offer the potential for regulation of transcription and expression are useful in instances where repression of gene transcription is sought. The adenoviral gene expression cassette may comprise a transcription termination sequence; specific embodiments of which are the bovine growth hormone termination/polyadenylation signal (bGHpA) or the short synthetic polyA signal (SPA) of 50 nucleotides in length defined as follows: AATAAAAGATCTTTATTTTCATTAGATCTGTGTGTTGGTTTTTTGTGTG (SEQ ID NO:3). A leader or signal peptide may also be incorporated into the transgene. In specific embodiments, the leader is derived from the tissue-specific plasminogen activator protein, tPA.

The recombinant adenovirus may be administered alone, or as part of a prime/boost-type administration regimen. In this scenario, an individual is first administered a priming dose of a viral (or polynucleotide) vehicle comprising nucleic acid encoding an HIV antigen and, following some period of time, administered a boosting dose of a viral (or polynucleotide) vehicle comprising nucleic acid encoding an HIV antigen; provided that either the priming or boosting administration employs an adenoviral vehicle. The priming dose effectively primes the immune response so that, upon subsequent identification of the antigen(s) in the circulating immune system, the immune response is capable of immediately recognizing and responding to the antigen(s) within the host. Preferably, the viral vehicles of the priming and boosting administrations are different in order to evade any host immunity directed against the first delivered vehicle. Selection of the alternate viral vehicle is not critical to the success of the methods disclosed herein. Any vehicle capable of delivering the antigen and accomplishing sufficient expression of said antigen such that a cellular-mediated immune response is elicited should be sufficient to prime or boost the adenovirally-mediated administration. A mixed modality prime and boost inoculation scheme will result in an enhanced immune response, particularly where there is pre-existing anti-vector immunity. Prime-boost administrations typically involve priming the subject (by viral vector, plasmid, protein, etc.) at least one time, allowing a predetermined length of time to pass, and then boosting (bay viral vector, plasmid, protein, etc.). Multiple primings, typically 1-4, are usually employed, although more may be used. The length of time between priming and boost may typically vary from about four months to a year, albeit other time frames may be used as one of ordinary skill in the art will appreciate. The follow-up or boosting administration may as well be repeated at selected time intervals.

Prime-boost regimens can employ different adenoviral serotypes, virus of different origin, viral vector/protein combinations, and combinations of viral and polynucleotide administrations. One example of such a protocol would be a priming dose(s) comprising a recombinant adenoviral vector of a first serotype followed by a boosting dose comprising a recombinant adenoviral vector of a second and different serotype. An example of such an embodiment would comprise the administration of a priming dose(s) comprising a recombinant adenoviral vector of serotype 5 followed up by a subsequent boosting dose(s) comprising a recombinant adenoviral vector of serotype 6; International Application No. PCT/US03/07727, filed Mar. 12, 2003; which is hereby incorporated by reference. An alternative embodiment would comprise the use of different viral vehicles of diverse origin in the prime and boost administrations, provided that at least either the prime and/or boost administration use an adenovirus vehicle. Examples of different viral vehicles include but are not limited to adeno-associated virus (“AAV”; see, e.g., Samulski et al., 1987 J. Virol. 61:3096-3101; Samulski et al., 1989 J. Virol. 63:3822-3828); retrovirus (see, e.g., Miller, 1990 Human Gene Ther. 1:5-14; Ausubel et al., Current Protocols in Molecular Biology); pox virus (including but not limited to replication-impaired NYVAC, ALVAC, TROVAC and MVA vectors, see, e.g., Panicali & Paoletti, 1982 Proc. Natl. Acad. Sci. USA 79:4927-31; Nakano et al. 1982 Proc. Natl. Acad. Sci. USA 79: 1593-1596; Piccini et al., In Methods in Enzymology 153:545-63 (Wu & Grossman, eds., Academic Press, San Diego); Sutter et al., 1994 Vaccine 12:1032-40; Wyatt et al., 1996 Vaccine 15:1451-8; and U.S. Pat. Nos. 4,603,112; 4,769,330; 4,722,848; 4,603,112; 5,110,587; 5,174,993; and 5,185,146); and alpha virus (see, e.g., WO 92/10578; WO 94/21792; WO 95/07994; and U.S. Pat. Nos. 5,091,309 and 5,217,879). Prime-boost protocols exploiting adenoviral and pox viral vectors for delivery of HIV antigens are discussed in International Application No. PCT/US03/07511, filed Mar. 12, 2003; which is hereby incorporated by reference. An alternative to the above immunization schemes would be to employ polynucleotide administrations (including but not limited to “naked DNA” or facilitated polynucleotide delivery) in conjunction with an adenoviral prime and/or boost; see, e.g., Wolff et al., 1990 Science 247:1465, and the following patent publications: U.S. Pat. Nos. 5,580,859; 5,589,466; 5,739,118; 5,736,524; 5,679,647; WO 90/11092 and WO 98/04720. Another alternative would be to employ recombinant protein administration in a prime-boost scheme along with adenovirus.

Potential hosts/vaccinees/individuals include but are not limited to primates and especially humans and non-human primates, and include any non-human mammal of commercial or domestic veterinary importance.

Compositions comprising the recombinant viral vectors may contain physiologically acceptable components, such as buffer, normal saline or phosphate buffered saline, sucrose, other salts and polysorbate. In certain embodiments, the formulation has: 2.5-10 mM TRIS buffer, preferably about 5 mM TRIS buffer, 25-100 mM NaCl, preferably about 75 mM NaCl; 2.5-10% sucrose, preferably about 5% sucrose; 0.01-2 mM MgCl₂; and 0.001%-0.01% polysorbate 80 (plant derived). The pH should range from about 7.0-9.0, preferably about 8.0. One skilled in the art will appreciate that other conventional vaccine excipients may also be used in the formulation. In specific embodiments, the formulation contains 5 mM TRIS, 75 mM NaCl, 5% sucrose, 1 mM MgCl₂, 0.005% polysorbate 80 at pH 8.0. This has a pH and divalent cation composition which is near the optimum for virus stability and minimizes the potential for adsorption of virus to glass surface. It does not cause tissue irritation upon intramuscular injection. It is preferably frozen until use.

The amount of viral particles in the vaccine composition to be introduced into a vaccine recipient will depend on the strength of the transcriptional and translational promoters used and on the immunogenicity of the expressed gene product. In general, an immunologically or prophylactically effective dose of 1×10⁷ to 1×10¹² particles and preferably about 1×10¹⁰ to 1×10¹¹ particles is administered directly into muscle tissue. Subcutaneous injection, intradermal introduction, impression through the skin, and other modes of administration such as intraperitoneal, intravenous, or inhalation delivery are also contemplated. Parenteral administration, such as intravenous, intramuscular, subcutaneous or other means of administration of additional agents able to potentiate or broaden the immune response (e.g., interleukin-12), concurrently with or subsequent to parenteral introduction of the vaccine compositions of this invention is also advantageous.

The following non-limiting Examples are presented to illustrate the present invention.

### EXAMPLE 1

Construction of an Ad5 Pre-Adenovirus Plasmid Containing the SIV Gag Gene

A. Construction of Adenoviral Shuttle Vector

The SIV gag sequence was originally isolated from strain mac239 (Kestler et al., 1990 Science 248:1109-1112). Codon-optimized DNA sequence (SEQ ID NO: 1) was chemically synthesized and cloned into pV1R-CMVI-SIVgag(Egan et al., 2000 J. Virol. 74:7485-7495). SIV gag DNA was isolated from plasmid pV1R-CMVI-SIVgag by digestion using restriction endonuclease BglII. The BglII fragment was then gel purified and ligated into the BglII site in plasmid pMA1 (also referred to as MRKpde1E1+CMVmin+BGHpA(str.)); a plasmid containing Ad5 sequence from base pair (“bp”) 1 to 5792 with a deletion of E1 sequences from bp 451 to 3510, and an HCMV promoter and BGHpA inserted into the E1 deletion in an E1 parallel orientation with a unique BglII site separating them. This process generated the Ad5 pre-plasmid pMA1-hCMV8-SIVgag, which was later renamed MRKpA1-hCMV8-SIVgag. The genetic structure of MRKpA1-hCMV8-SIVgag (pMA1-hCMV8-SIVgag) was verified by restriction enzyme and DNA sequencing.

B. Construction of Pre-Adenovirus Plasmid

The shuttle plasmid MRKpA1-hCMV8-SIVgag (pMA1-hCMV8-SIVgag) was digested with restriction enzymes SgrAI and BstZ17I and then co-transformed into E. coli strain BJ5183 with linearized (ClaI-digested) Ad5 backbone plasmid, MRKpAd(E1-/E3-)ClaI. The resulting MRKpAd-hCMV8-SIVgag was recovered from BJ1583 and re-transformed into competent E. coli Stb12 for large-scale production. The genetic structure of MRKpAd-hCMV8-SIVgag was verified by restriction enzyme digestion. ELISA and western results confirmed SIV gag gene expression.

### EXAMPLE 2

Construction of an Ad5 Pre-Adenovirus Plasmid Containing the SIV nef Gene

A. Creation of SIV nefG2A Mutation and Construction of Adenoviral Shuttle Vector

The SIV nef sequence was originally isolated from strain mac251 (Kestler, et al., 1988 Nature 331:619-622). Codon-optimized DNA sequence (SEQ ID NO: 2) was chemically synthesized and cloned into pA1-To-SIVnef. Plasmid pA1-To-SIVnef utilizes the human CMV promoter regulated by the tetracycline operator (To) and the bovine growth hormone transcription terminator/polyadenylation signal as expression regulatory elements for the SIV nef gene. The second codon GGT for glycine (G) of SIV nef was converted to GCC for alanine (A) by PCR amplification using primers containing GCC and BclI site at each end. The new gene is designated nefGCC (new codon) or nefG2A (amino acid change). The nef gene was PCR amplified using primers containing GCC for the second codon position. The PCR product was digested by BclI, gel purified and ligated into the BglII restriction endonuclease site (cohesive ends of BclI and BglII are compatible) in the MRKAd5 shuttle plasmid MRK2, generating plasmid MRK2-hCMV-SIVnefGCC. The genetic structure of the plasmid was verified by DNA sequencing and restriction enzyme digestion.

B. Construction of Pre-Adenovirus Plasmid

The shuttle plasmid MRK-hCMV-SIVnefGCC was digested with restriction enzymes BstZ17I and SgrAI and then co-transformed into E. coli strain BJ5183 with linearized (ClaI-digested) Ad5 backbone plasmid, pHVE3. The resulting MRKpAd-E3-hCMV-SIVnef(GCC) was recovered from BJ1583 and re-transformed into competent E. coli Stb12 for large-scale production. The genetic structure of the pre-plasmid MRKpAd-E3-hCMV-SIVnef(GCC) was verified by restriction enzyme digestion. Western results confirmed SIV nefGCC gene expression.

### EXAMPLE 3

Generation of Research-Grade Recombinant Adenovirus

To prepare virus for pre-clinical animal studies, the pre-adenovirus plasmid was rescued as infectious virions in PER.C6® adherent monolayer cell culture. To rescue infectious virus MRKAd5SIVgag, 30 μg of MRKpAd-hCMV8-SIVgag was digested with restriction enzyme PacI (New England Biolabs) and transfected into a T75 flask of PER.C6® cells using the GenePorter2 kit (GTS, Gene Therapy Systems, Inc.). To rescue infectious virus MRKAd5SIVnefGCC, 30 μg of pre-adenovirus plasmid MRKpAd-E3-hCMV-SIVnef(GCC) was digested with restriction enzyme PacI (New England Biolabs) and transfected into a T75 flask of PER.C6® cells using the calcium phosphate co-precipitation technique (Cell Phect Transfection Kit, Amersham Pharmacia Biotech Inc.). PacI digestion released the viral genome from plasmid sequences allowing viral replication to occur after entry into PER.C6®cells. Infected cells and media were harvested after complete viral cytopathic effect (CPE) was observed. The virus stock was amplified by multiple passages in PER.C6® adherent monolayer cell culture. At the final passage, virus was purified from the cell pellet by CsCl ultracentrifugation and characterized. The virus quantity was determined using analytical assays that quantify the viral genomes for viral particles. The viral infectivity was determined by Tissue Culture Infectious Dose 50% (TCID₅₀) assay. The identity and purity of the purified virus was confirmed by restriction endonuclease (HindIII+PacI) analysis of purified viral DNA. For restriction analysis, digested viral DNA was end-labeled with P³³-dATP, size-fractionated by agarose gel electrophoresis, and visualized by autoradiography. The gene expression for SIV gag and nefGCC (G2A) was monitored by ELISA or western with materials collected from virus infected mammalian cells grown in vitro. The stocks of MRKAd5SIVgag and MRKAd5SIVnefGCC (MRKAd-E3-hCMV-SIVnef(GCC)) were used in immunological evaluation in mice and rhesus monkeys.

### EXAMPLE 4

Construction of an Ad6 Pre-Adenovirus Plasmid Containing the SIV Gag Gene

The MRKAd shuttle plasmid pMRKhCMVSIVgagbGH (also referred to as MRKpA1-hCMV8-SIVgag or pMA1-hCMV8-SIVgag) that was used for the generation of MRKAd pre-plasmid carrying SIV gag gene was used to generate the corresponding MRKAd6 pre-plasmid. The shuttle plasmid pMRKhCMVSIVgagbGH was digested with EcoRI and StuI and then co-transformed into E. coli strain BJ5183 with linearized (ClaI-digested) Ad6 backbone plasmid, pMRKAd6E1-. The recovered plasmid was re-transformed into competent E. coli Stb12 for large-scale production. The genetic structure of the pre-plasmid pMRKAd6E1-hCMVSIVgagbGH was verified by restriction enzyme digestion.

### EXAMPLE 5

Construction of Ad6 Pre-Adenovirus Plasmid Containing SIV nefGCC Gene

The MRKAd5 shuttle plasmid, pMRKhCMVSIVnef(G2A) (also referred to as MRK2-hCMV-SIVnef(GCC), which was used for the generation of MRKAd5 pre-plasmid carrying SIV nef(GCC), was used to generate the corresponding MRKAd6 pre-plasmid. The shuttle plasmid pMRKhCMVSIVnef(G2A) was digested with EcoRI and BstXI and then co-transformed into E. coli strain BJ5183 with linearized (ClaI-digested) Ad6 backbone plasmid, pMRKAd6E1-. The recovered plasmid was then re-transformed into competent E. coli Stb12 for large-scale production. The genetic structure of the pre-plasmid pMRKAd6E1-hCMVSIVnefbGH (GCC or G2A) was verified by restriction enzyme digestion.

### EXAMPLE 6

Generation of Research-Grade Recombinant MRKAd6 gag and nef

To prepare virus for pre-clinical immunogenicity studies, the pre-adenovirus plasmids pMRKAd6E1-hCMVSIVgagbGH and pMRKAd6E1-hCMVSIVnefbGH were rescued as infectious virions in PER.C6® adherent monolayer cell culture. To rescue infectious virus, 30 μg of pMRKAd6E1-hCMVSIVgagbGH or pMRKAd6E1-hCMVSIVnefbGH were partially digested with restriction enzyme PacI (New England Biolabs) and transfected into T75 flask of PER.C6® cells using the calcium phosphate co-precipitation technique (Cell Phect Transfection Kit, Amersham Pharmacia Biotech Inc.). pMRKAd6E1-hCMVSIVgagbGH and pMRKAd6E1-hCMVSIVnefbGH each contain three PacI restriction sites; one at each ITR and one located in early region 3. Digestion conditions which favored the linearization of the pre-Ad plasmids (digestion at only one of the three PacI sites) were used since the release of only one ITR is required to allow the initiation of viral DNA replication after entry into PER.C6®cells. Infected cells and media were harvested after complete viral cytopathic effect (CPE) was observed. The virus stock was amplified by multiple passages in PER.C6® cells. At the final passage, virus was purified from the cell pellet by CsCl ultracentrifugation and characterized. The virus quantity was determined using analytical assays that quantify the viral genomes for viral particles. The viral infectivity was determined by Tissue Culture Infectious Dose 50% (TCID₅₀) assay. The identity and purity of the purified virus was confirmed by restriction endonuclease (HindIII+PacI) analysis of purified viral DNA. For restriction analysis, digested viral DNA was end-labeled with P³³-dATP, size-fractionated by agarose gel electrophoresis, and visualized by autoradiography. The gene expression for SIV gag and nef (GCC or G2A) was monitored by ELISA or western with materials collected from virus infected mammalian cells grown in vitro. The stocks of MRKAd6hCMVSIVgagbGH and MRKAd6hCMVSIVnefbGH (GCC or G2A) were used in immunological evaluation in mice and rhesus monkeys.

### EXAMPLE 7

Drug Formulation

Fresh solution of the compound (N-1-(7-{[(4-fluorobenzyl)amino]carbonyl}-8-hydroxy-1,6-naphthyridin-5-yl)-N-1-,N-2-,N-2-trimethylethanediamide, disclosed in US Application Serial No. US 2003/0055071, published Mar. 20, 2003) was formulated on a weekly basis in the following manner. Compound was weighed out accurately and solubilized in distilled, deionized water at a concentration of 5.24 mg/mL. Solubilization is complete when the liquid is clear and contains no visible compound particulates.

### EXAMPLE 8

Administration of Virus, Test Drug and Vaccines

The study consisted of four (4) cohorts of mamuA01(+) rhesus macaques. At day 0, all cohorts were infected with SIVmac239 intrarectally. The virus was prepared in the following manner. The virus was diluted in 10% fetal bovine serum/RPMI 1640 cell culture media to a final concentration of 3.2×10⁻⁵ TCID50 per mL. 1-mL volumes were filled into separate syringes for intrarectal administration. At day 30, animals of cohort 1 and 3 were initiated on BID doses of (N-1-(7-{[(4-fluorobenzyl)amino]carbonyl}-8-hydroxy-1,6-naphthyridin-5-yl)-N-1-,N-2-,N-2-trimethylethanediamide. Each monkey was dosed at 20.98 mg/kg/day of the compound which was delivered via a nasal-gastric tube. At day 122 and 150, cohorts 1 and 2 were given intramuscular doses of a cocktail of 5×10¹⁰ vp MRKAd5-SIVgag+5×10¹⁰ vp MRKAd5-SIVnef followed by a booster with a cocktail of 5×10¹⁰ vp MRKAd6-SIVgag+5×10¹⁰ vp MRKAd6-SIVnef at day 234. In all cases, the total dose of each vaccine was suspended in 1 mL of buffer. The macaques were anesthetized (ketamine/xylazine) and the vaccines were delivered i.m. in 0.5-mL aliquots into both deltoid muscles using tuberculin syringes (Becton-Dickinson). Cohort 4 received neither the drug nor immunizations. Plasma, sera and peripheral blood mononuclear cells (PBMC) were prepared from blood samples collected at several time points during the immunization regimen. All animal care and treatment were in accordance with standards approved by the Institutional Animal Care and Use Committee according to the principles set forth in the Guide for Care and Use of Laboratory Animals, Institute of Laboratory Animal Resources, National Research Council.

### EXAMPLE 9

ELISPOT Assay

The IFN-γ ELISPOT assays for rhesus macaques were conducted following a previously described protocol (Allen et al., 2001 J. Virol. 75(2):738-749), with some modifications. For antigen-specific stimulation, a peptide pool was prepared from 20-aa peptides that encompass the entire HIV-1 gag sequence with 10-aa overlaps (Synpep Corp., Dublin, Calif.). To each well, 50 μL of 2-4×10⁵ peripheral blood mononuclear cells (PBMCs) were added; the cells were counted using Beckman Coulter Z2 particle analyzer with a lower size cut-off set at 80 femtoliters (“fL”). Either 50 μL of media or the gag peptide pool at 8 μg/mL concentration per peptide was added to the PBMC. The samples were incubated at 37° C., 5% CO₂ for 20-24 hrs. Spots were developed accordingly and the plates were processed using custom-built imager and automatic counting subroutine based on the ImagePro platform (Silver Spring, Md.); the counts were normalized to 10⁶ cell input.

### EXAMPLE 10

Intracellular Cytokine Staining

To 1 ml of 2×10⁶ PBMC/mL in complete RPMI media (in 17×100 mm round bottom polypropylene tubes (Sarstedt, Newton, N.C.)), anti-hCD28 (clone L293, Becton-Dickinson) and anti-hCD49d (clone L25, Becton-Dickinson) monoclonal antibodies were added to a final concentration of 1 μg/mL. For gag-specific stimulation, 10 μL of the peptide pool (at 0.4 mg/mL per peptide) were added. The tubes were incubated at 37° C. for 1 hr., after which 20 μL of 5 mg/mL of brefeldin A (Sigma) were added. The cells were incubated for 16 hr at 37° C., 5% CO₂, 90% humidity. 4 mL cold PBS/2% FBS were added to each tube and the cells were pelleted for 10 min at 1200 rpm. The cells were re-suspended in PBS/2% FBS and stained (30 min, 4° C.) for surface markers using several fluorescent-tagged mAbs: 20 μL per tube anti-hCD3-APC, clone FN-18 (Biosource); 20 μL anti-hCD8-PerCP, clone SK1 (Becton Dickinson, Franklin Lakes, N.J.); and 20 μL anti-hCD4-PE, clone SK3 (Becton Dickinson). Sample handling from this stage was conducted in the dark. The cells were washed and incubated in 750 μL 1×FACS Perm buffer (Becton Dickinson) for 10 min at room temperature. The cells were pelleted and re-suspended in PBS/2% FBS and 0.1 μg of FITC-anti-hIFN-γ, clone MD-1 (Biosource) was added. After 30 min incubation, the cells were washed and re-suspended in PBS. Samples were analyzed using all four color channels of the Becton Dickinson FACSCalibur instrument. To analyze the data, the low side- and forward-scatter lymphocyte population was initially gated; a common fluorescence cut-off for cytokine-positive events was used for both CD4+ and CD8+ populations, and for both mock and gag-peptide reaction tubes of a sample.

### EXAMPLE 11

Viral Load Determination

Viral load was determined from EDTA-treated plasma by an assay conducted at Consolidated Laboratory Services, Van Nuys, Calif. referred to as SIV Real-time RNA Level using the ABI Prism 7700 sequence detection system (Leutenegger, et al., 2001 AIDS Res. Human Retro. 17(3):243-51; Hofmann-Lehmann). This real-time assay demonstrated to be accurate, sensitive and reproducible over eight orders of magnitude, permitting effective characterization of viral load during the course of the study. This test detects SIV viral load specifically not HIV. Linearity ranged from 10¹ to 10⁹ copies/mL.

### EXAMPLE 12

Results

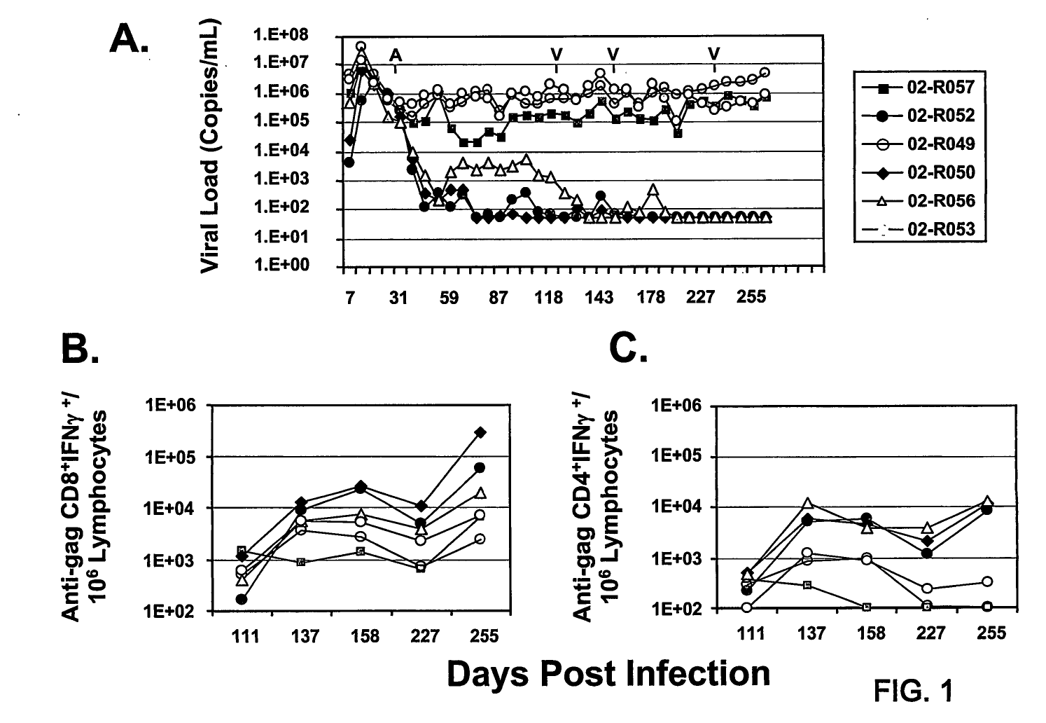

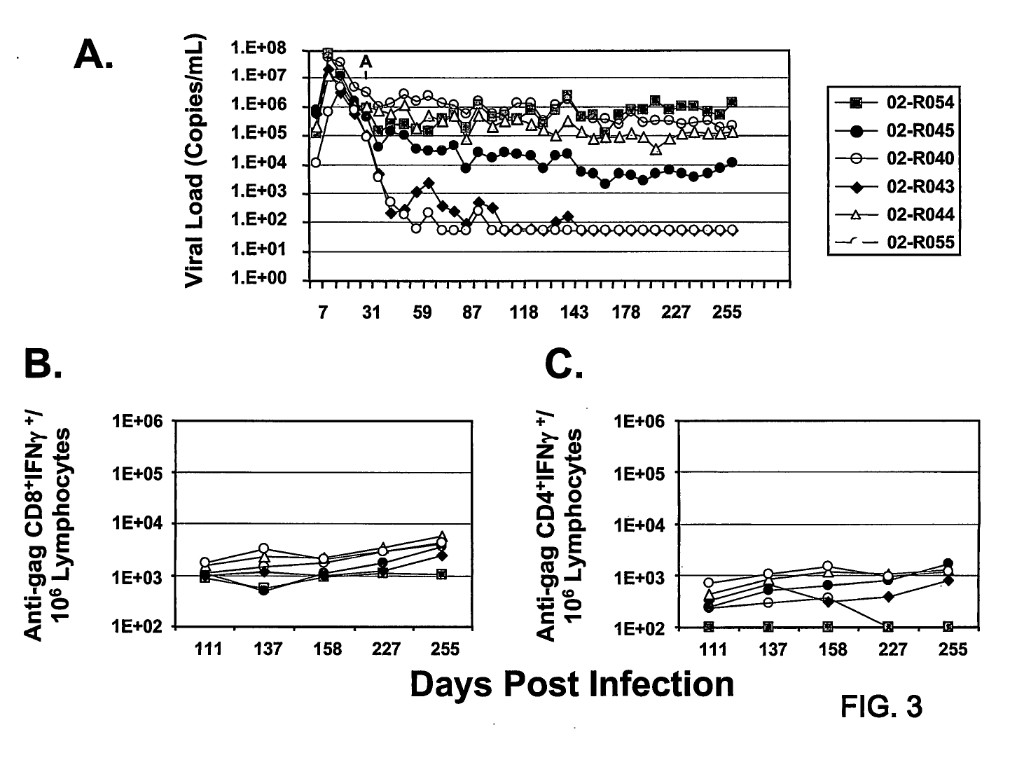

All animals in the study showed peak levels of viral replication (3×10⁶ to 9×10⁸ viral copies/mL) within the first 17 days of infection with SIVmac239 (FIG. 1A, 2A, 3A, 4A). In cohort 1 (FIG. 1A), 3 of 6 animals responded to drug treatment which was initiated at day 30; viral loads dropped 3 or more orders of magnitude to baseline levels. In cohort 3 (FIG. 3A), 2 of 6 animals had responded strongly to drug therapy with viral loads dropping to baseline levels.

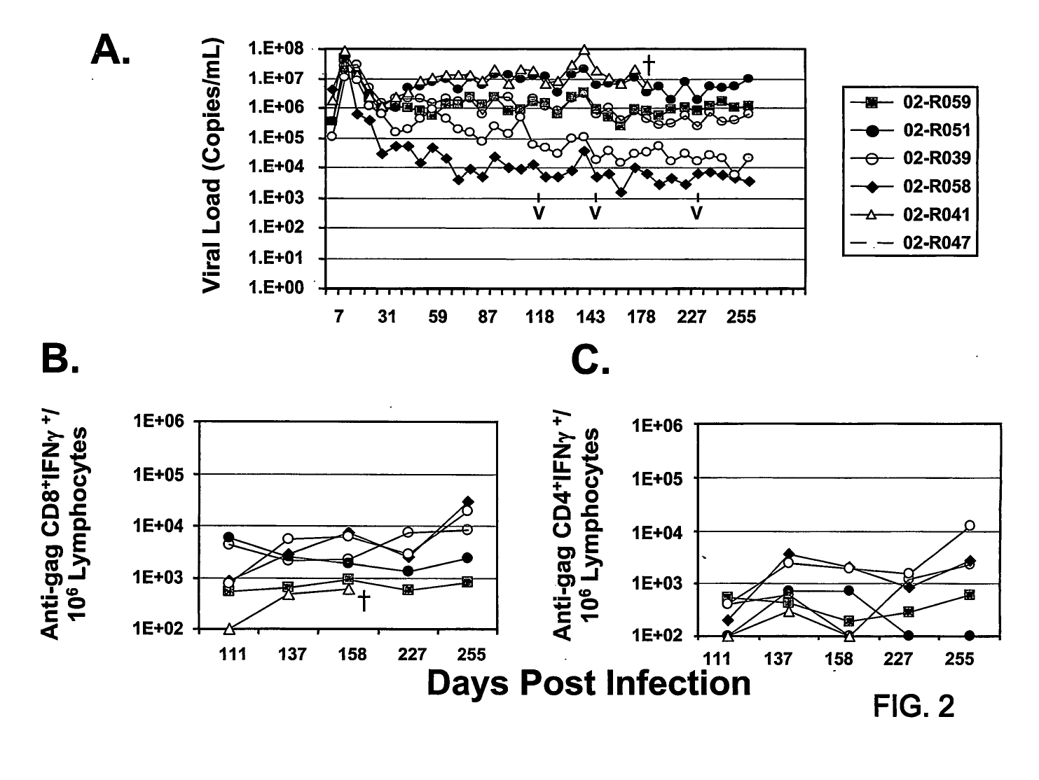

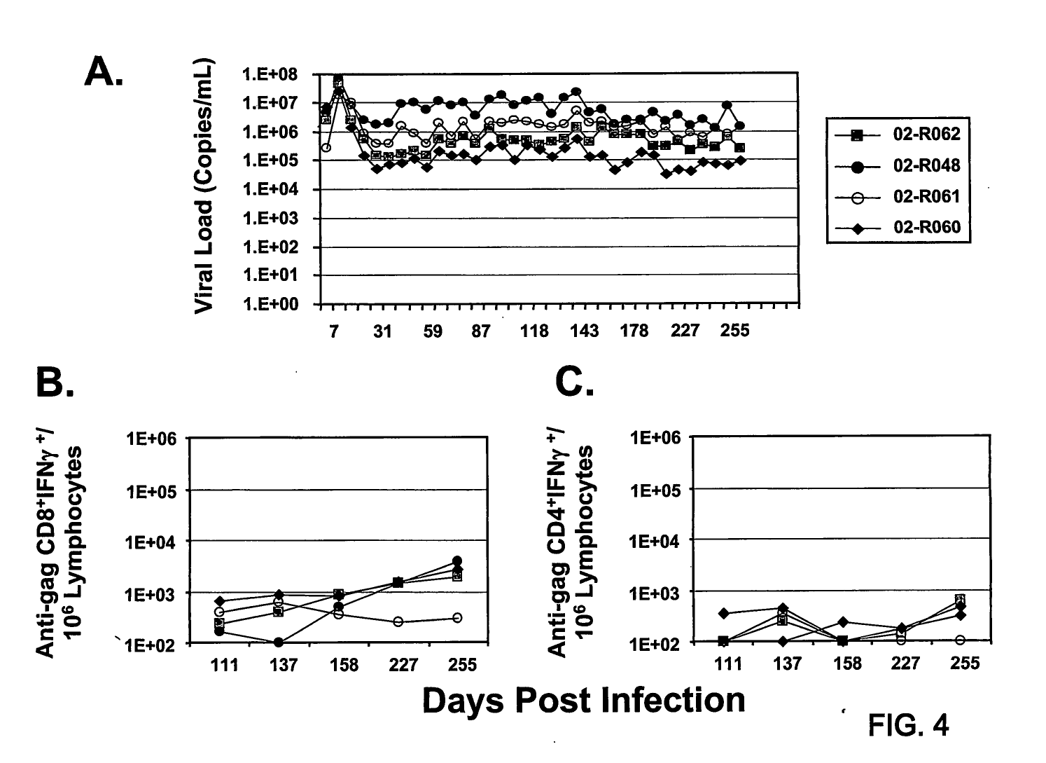

At day 122 and day 150, cohorts 1 and 2 received immunizations of MRKAd-SIVgag plus MRKAd5-SIVnef followed by a dosing at day 234 with a mixture of MRKAd6-SIVgag plus MRKAd6-SIVnef. Anti-gag T cell responses were evaluated using intracellular cytokine staining at day 111, 137, 158 and 255. The results are summarized in FIGS. 1B, 1C, 2B, 2C, 3B, 3C, 4B, and 4C. In cohort 1 (FIGS. 1B, 1C), immunization with MRKAd5-based vaccine induced a dramatic increase in both gag-specific cytotoxic CD8+ and helper CD4+ responses in animals 02-R052, 02-R050 and 02-R056 (>10-fold). All 3 animals had drug-induced control of their viral load levels. Increases in gag-specific T cell responses were also apparent in these animals upon immunization with the MRKAd6-based vaccine. The only other animal that exhibited an increase in CD8+ and CD4+ responses was 02-R053; the animal did not show control of viral load in response to continued drug therapy. However, the increase in T cell responses did not sustain upon administration of the MRKAd6 follow-up vaccine. In cohort 2 (FIG. 2B, 2C) for which no drug treatment was given, 2 animals (02-R058, 02-R047) appear to spontaneously exhibit virus control better than the rest of the animals in the cohort and these 2 animals showed a notable increase in both CD8+ and CD4+ responses against gag after the MRKAd5 and MRKAd6 immunization. As expected there were no significant fluctuations in T cell responses in cohort 3 and 4. In general, the levels of T cell responses are highest in cohort 1 followed by cohort 2, cohort 3 and finally cohort 4. Similar trends were observed for anti-nef T cell responses in all four cohorts (data not shown).

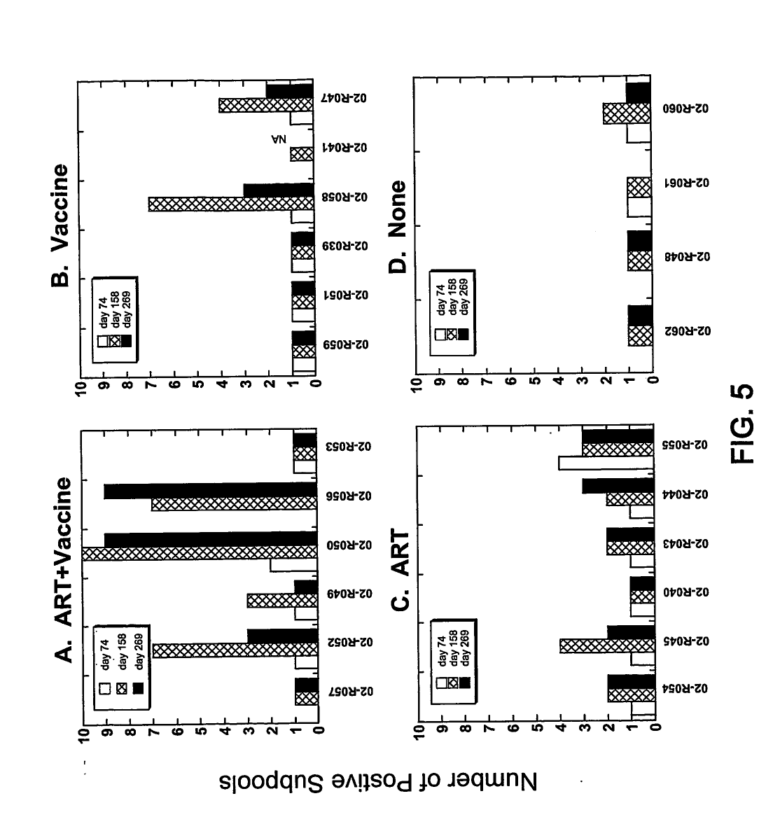

The breadth of the T cell response was also evaluated in an ELISPOT assay by dividing the gag peptide pool into 10 smaller subpools. Each represents about 50-aa segment of the protein originating from the N-terminus to the C-terminus. PBMCs from animals were tested against the subpools at day 74, day 158 and day 269. FIG. 5 shows the number of subpools to which a positive antigen-specific response was detected for each animal at a given time point. The broadest T cell responses were observed in cohort 1, specifically in the animals (02-R052, 02-R050, 02-R056) that exhibited drug-induced virus control and strongest immune response to the vaccine.

The findings support the concept that adenoviral-mediated immunization of infected individuals exhibiting controlled viremia can provide very high levels of both virus-specific CD8+ and CD4+ T cell responses of a very broad nature. This method of eliciting an enhanced immune response should assist infected individuals in maintaining low viral load and, thus, offers the prospect of reducing individual dependency on antiviral therapy.

What is claimed:

1. A method for eliciting a cellular-mediated immune response against human immunodeficiency virus (“HIV”) in an individual infected with HIV, which comprises:
administering to an HIV-infected individual a recombinant, replication-defective adenovirus comprising nucleic acid encoding an HIV-1 antigen;
wherein said individual has experienced, prior to the administration, a reduction in HIV viral copy number.

2. A method in accordance with claim 1 wherein the reduction in HIV viral copy number was due at least in part to treatment with antiviral agents.

3. A method in accordance with claim 2 wherein the antiviral agents comprise one or more of the following: a protease inhibitor, an inhibitor of reverse transcriptase, and an integrase inhibitor.

4. A method in accordance with claim 2 wherein the antiviral agents comprise a combination of protease inhibitors and inhibitors of reverse transcriptase

5. A method in accordance with claim 1 which comprises administering and readministering the adenovirus to the individual.

6. A method in accordance with claim 1 which further comprises administering an adenovirus of an alternative serotype comprising nucleic acid encoding an HIV antigen.

7. A method in accordance with claim 1 which further comprises administering a virus of different viral origin comprising nucleic acid encoding an HIV antigen.

8. A method in accordance with claim 1 where the antigen is derived from HIV gag.

9. A method in accordance with claim 1 where the antigen is derived from HIV nef.

10. A method in accordance with claim 1 where the antigen is derived from HIV pol.

11. A method in accordance with claim 1 where the antigen is derived from HIV env.

12. A method in accordance with claim 1 which further comprises administering a polynucleotide composition comprising nucleic acid encoding an HIV antigen.
